SEQUENCE LISTING (3 sequences)

SEQ ID NO: 1 - protein, 1404 aa, Homo sapiens
MAWKTLPIYLLLLLSVFVIQQVSSQDLSSCAGRCGEGYSRDATCNCDYNCQHYMECCPDFKRVCTAELSCKGRCFESFERGRECDCDAQCKKYDKCCPDYESFCAEVHNPTSPPSSKKAPPPSGASQTIKSTTKRSPKPPNKKKTKKVIESEEITEEHSVSENQESSSSSSSSSSSSTIRKIKSSKNSAANRELQKKLKVKDNKKNRTKKKPTPKPPVVDEAGSGLDNGDFKVTTPDTSTTQHNKVSTSPKITTAKPINPRPSLPPNSDTSKETSLTVNKETTVETKETTTTNKQTSTDGKEKTTSAKETQSIEKTSAKDLAPTSKVLAKPTPKAETTTKGPALTTPKEPTPTTPKEPASTTPKEPTPTTIKSAPTTPKEPAPTTTKSAPTTPKEPAPTTTKEPAPTTPKEPAPTTTKEPAPTTTKSAPTTPKEPAPTTPKKPAPTTPKEPAPTTPKEPTPTTPKEPAPTTKEPAPTTPKEPAPTAPKKPAPTTPKEPAPTTPKEPAPTTTKEPSPTTPKEPAPTTTKSAPTTTKEPAPTTTKSAPTTPKEPSPTTTKEPAPTTPKEPAPTTPKKPAPTTPKEPAPTTPKEPAPTTTKKPAPTTPKEPAPTTPKETAPTTPKKLTPTTPEKLAPTTPEKPAPTTPEELAPTTPEEPTPTTPEEPAPTTPKAAAPNTPKEPAPTTPKEPAPTTPKEPAPTTPKETAPTTPKGTAPTTLKEPAPTTPKKPAPKELAPTTTKEPTSTTCDKPAPTTPKGTAPTTPKEPAPTTPKEPAPTTPKGTAPTTLKEPAPTTPKKPAPKELAPTTTKGPTSTTSDKPAPTTPKETAPTTPKEPAPTTPKKPAPTTPETPPPTTSEVSTPTTTKEPTTIHKSPDESTPELSAEPTPKALENSPKEPGVPTTKTPAATKPEMTTTAKDKTTERDLRTTPETTTAAPKMTKETATTTEKTTESKITATTTQVTSTTTQDTTPFKITTLKTTTLAPKVTTTKKTITTTEIMNKPEETAKPKDRATNSKATTPKPQKPTKAPKKPTSTKKPKTMPRVRKPKTTPTPRKMTSTMPELNPTSRIAEAMLQTTTRPNQTPNSKLVEVNPKSEDAGGAEGETPHMLLRPHVFMPEVTPDMDYLPRVPNQGIIINPMLSDETNICNGKPVDGLTTLRNGTLVAFRGHYFWMLSPFSPPSPARRITEVWGIPSPIDTVFTRCNCEGKTFFFKDSQYWRFTNDIKDAGYPKPIFKGFGGLTGQIVAALSTAKYKNWPESVYFFKRGGSIQQYIYKQEPVQKCPGRRPALNYPVYGETTQVRRRRFERAIGPSQTHTIRIQYSPARLAYQDKGVLHNEVKVSILWRGLPNVVTSAISLPNIRKPDGYDYYAFSKDQYYNIDVPSRTARAITTRSGQTLSKVWYNCP

SEQ ID NO: 2 - DNA, 5041 bp, Homo sapiens
gcggccgcgactattcggtacctgaaaacaacgatggcatggaaaacacttcccatttacctgttgttgctgctgtctgttttcgtgattcagcaagtttcatctcaagatttatcaagctgtgcagggagatgtggggaagggtattctagagatgccacctgcaactgtgattataactgtcaacactacatggagtgctgccctgatttcaagagagtctgcactgcggagctttcctgtaaaggccgctgctttgagtccttcgagagagggagggagtgtgactgcgacgcccaatgtaagaagtatgacaagtgctgtcccgattatgagagtttctgtgcagaagtgcataatcccacatcaccaccatcttcaaagaaagcacctccaccttcaggagcatctcaaaccatcaaatcaacaaccaaacgttcacccaaaccaccaaacaagaagaagactaagaaagttatagaatcagaggaaataacagaagaacattctgtttctgaaaatcaagagtcctcctcctcctcctcctcttcctcttcttcttcaacaatttggaaaatcaagtcttccaaaaattcagctgctaatagagaattacagaagaaactcaaagtaaaagataacaagaagaacagaactaaaaagaaacctacccccaaaccaccagttgtagatgaagctggaagtggattggacaatggtgacttcaaggtcacaactcctgacacgtctaccacccaacacaataaagtcagcacatctcccaagatcacaacagcaaaaccaataaatcccagacccagtcttccacctaattctgatacatctaaagagacgtctttgacagtgaataaagagacaacagttgaaactaaagaaactactacaacaaataaacagacttcaactgatggaaaagagaagactacttccgctaaagagacacaaagtatagagaaaacatctgctaaagatttagcacccacatctaaagtgctggctaaacctacacccaaagctgaaactacaaccaaaggccctgctctcaccactcccaaggagcccacgcccaccactcccaaggagcctgcatctaccacacccaaagagcccacacctaccaccatcaagtctgcacccaccacccccaaggagcctgcacccaccaccaccaagtctgcacccaccactcccaaggagcctgcacccaccaccaccaaggagcctgcacccaccactcccaaggagcctgcacccaccaccaccaaggagcctgcacccaccaccaccaagtctgcacccaccactcccaaggagcctgcacccaccacccccaagaagcctgccccaactacccccaaggagcctgcacccaccactcccaaggagcctacacccaccactcccaaggagcctgcacccaccaccaaggagcctgcacccaccactcccaaagagcctgcacccactgcccccaagaagcctgccccaactacccccaaggagcctgcacccaccactcccaaggagcctgcacccaccaccaccaaggagccttcacccaccactcccaaggagcctgcacccaccaccaccaagtctgcacccaccactaccaaggagcctgcacccaccactaccaagtctgcacccaccactcccaaggagccttcacccaccaccaccaaggagcctgcacccaccactcccaaggagcctgcacccaccacccccaagaagcctgccccaactacccccaaggagcctgcacccaccactcccaaggaacctgcacccaccaccaccaagaagcctgcacccaccgctcccaaagagcctgccccaactacccccaaggagactgcacccaccacccccaagaagctcacgcccaccacccccgagaagctcgcacccaccacccctgagaagcccgcacccaccacccctgaggagctcgcacccaccacccctgaggagcccacacccaccacccctgaggagcctgctcccaccactcccaaggcagcggctcccaacacccctaaggagcctgctccaactacccctaaggagcctgctccaactacccctaaggagcctgctccaactacccctaaggagactgctccaactacccctaaagggactgctccaactaccctcaaggaacctgcacccactactcccaagaagcctgcccccaaggagcttgcacccaccaccaccaaggagcccacatccaccacctctgacaagcccgctccaactacccctaaggggactgctccaactacccctaaggagcctgctccaactacccctaaggagcctgctccaactacccctaaggggactgctccaactaccctcaaggaacctgcacccactactcccaagaagcctgcccccaaggagcttgcacccaccaccaccaaggggcccacatccaccacctctgacaagcctgctccaactacacctaaggagactgctccaactacccccaaggagcctgcacccactacccccaagaagcctgctccaactactcctgagacacctcctccaaccacttcagaggtctctactccaactaccaccaaggagcctaccactatccacaaaagccctgatgaatcaactcctgagctttctgcagaacccacaccaaaagctcttgaaaacagtcccaaggaacctggtgtacctacaactaagactcctgcagcgactaaacctgaaatgactacaacagctaaagacaagacaacagaaagagacttacgtactacacctgaaactacaactgctgcacctaagatgacaaaagagacagcaactacaacagaaaaaactaccgaatccaaaataacagctacaaccacacaagtaacatctaccacaactcaagataccacaccattcaaaattactactcttaaaacaactactcttgcacccaaagtaactacaacaaaaaagacaattactaccactgagattatgaacaaacctgaagaaacagctaaaccaaaagacagagctactaattctaaagcgacaactcctaaacctcaaaagccaaccaaagcacccaaaaaacccacttctaccaaaaagccaaaaacaatgcctagagtgagaaaaccaaagacgacaccaactccccgcaagatgacatcaacaatgccagaattgaaccctacctcaagaatagcagaagccatgctccaaaccaccaccagacctaaccaaactccaaactccaaactagttgaagtaaatccaaagagtgaagatgcaggtggtgctgaaggagaaacacctcatatgcttctcaggccccatgtgttcatgcctgaagttactcccgacatggattacttaccgagagtacccaatcaaggcattatcatcaatcccatgctttccgatgagaccaatatatgcaatggtaagccagtagatggactgactactttgcgcaatgggacattagttgcattccgaggtcattatttctggatgctaagtccattcagtccaccatctccagctcgcagaattactgaagtttggggtattccttcccccattgatactgtttttactaggtgcaactgtgaaggaaaaactttcttctttaaggattctcagtactggcgttttaccaatgatataaaagatgcagggtaccccaaaccaattttcaaaggatttggaggactaactggacaaatagtggcagcgctttcaacagctaaatataagaactggcctgaatctgtgtattttttcaagagaggtggcagcattcagcagtatatttataaacaggaacctgtacagaagtgccctggaagaaggcctgctctaaattatccagtgtatggagaaatgacacaggttaggagacgtcgctttgaacgtgctataggaccttctcaaacacacaccatcagaattcaatattcacctgccagactggcttatcaagacaaaggtgtccttcataatgaagttaaagtgagtatactgtggagaggacttccaaatgtggttacctcagctatatcactgcccaacatcagaaaacctgacggctatgattactatgccttttctaaagatcaatactataacattgatgtgcctagtagaacagcaagagcaattactactcgttctgggcagaccttatccaaagtctggtacaactgtccttagactgatgagcaaaggaggagtcaactaatgaagaaatgaataataaattttgacactgaaaaacattttattaataaagaatattgacatgagtataccagtttatatataaaaatgtttttaaacttgacaatcattacactaaaacagatttgataatcttattcacagttgttattgtttacagaccatttaattaatatttcctctgtttattcctcctctccctcccattgcatggctcacacctgtaaaagaaaaaagaatcaaattgaatatatcttttaagaattcaaaactagtgtattcacttaccctagttcattataaaaaatatctaggcattgtggatataaaactgttgggtattctacaacttcaatggaaattattacaagcagattaatccctctttttgtgacacaagtacaatctaaaagttatattggaaaacatggaaatattaaaattttacacttttactagctaaaacataatcacaaagctttatcgtgttgtataaaaaaattaacaatataatggcaataggtagagatacaacaaatgaatataacactataacacttcatattttccaaatcttaatttggatttaaggaagaaatcaataaatataaaatataagcacatatttattatatatctaaggtatacaaatctgtctacatgaagtttacagattggtaaatatcacctgctcaacatgtaattatttaataaaactttggaacattaaaaaaataaattggaggcttaaaaaaaaaaaaaaaaa

SEQ ID NO: 3 - protein, 7 aa, Homo sapiens
KEPAPTT

(19) United States
(12) Patent Application Publication
(10) Pub. No.: US 2021/0260167 A1
(43) Pub. Date: Aug. 26, 2021

(54) LUBRICIN FOR USE IN WOUND HEALING

(71) Applicant: Lubris LLC, Framingham, MA (US)

(72) Inventors: Roman Krawetz, Calgary (CA); Saleem Abubacker, North York (CA); Jeffrey Alan Biernaskie, Calgary (CA); Tannin Avery Schmidt, Avon, CT (US)

(21) Appl. No.: 17/253,253

(22) PCT Filed: Jun. 21, 2019
(86) PCT No.: PCT/US2019/038472

§ 371 Date: Dec. 17, 2020

Related U.S. Application Data
(60) Provisional application No. 62/688,163, filed on Jun. 21, 2018.

(51) Int. Cl. A61K 38/39; A61K 31/167; A61K 31/245; A61K 31/7036; A61K 38/12; A61K 31/7048; A61K 31/46; A61K 31/18; A61K 31/351; A61K 31/5375; A61K 33/38; A61K 31/4709; A61K 9/00; A61P 27/02; A61P 17/02

(52) U.S. Cl. CPC A61K 38/39; A61P 17/02; A61K 31/245; A61K 31/7036; A61K 38/12; A61K 31/7048; A61K 31/46; A61K 31/18; A61K 31/351; A61K 31/5375; A61K 33/38; A61K 31/4709; A61K 9/0048; A61P 27/02; A61K 31/167

(57) ABSTRACT

The application relates to uses of the proteoglycan 4 protein, also known as lubricin, in wound healing and tissue regeneration, reducing scar formation, and promoting angiogenesis.

### CROSS-REFERENCE TO RELATED APPLICATIONS

This application claims priority to and the benefit of U.S. Provisional Patent Application No. 62/688,163 filed Jun. 21, 2018, the entire contents of which are incorporated by reference herein.

### FIELD OF THE INVENTION

This invention relates to new uses of the human glycoprotein PRG4, also known as lubricin. More particularly, it relates to using PRG4 as agent for promoting wound healing.

### BACKGROUND

The proteoglycan 4 gene (PRG4) encodes megakaryocyte stimulating factor (MSF) as well as highly glycosylated differently splice variants and glycoforms of “superficial zone protein” also known as lubricin. Superficial zone protein was first localized at the surface of explant cartilage from the superficial zone and identified in conditioned medium. PRG4, also known as lubricin, was first isolated from synovial fluid and demonstrated lubricating ability in vitro similar to synovial fluid at a cartilage-glass interface and in a latex-glass interface. It was later identified as a product of synovial fibroblasts, and its lubricating ability was discovered to be dependent on O-linked β (1-3) Gal-GalNAc oligosaccharides within a large mucin like domain of 940 amino acids encoded by exon 6. Lubricin molecules are differentially glycosylated and several naturally occurring splice variants have been reported. They are collectively referred to herein as PRG4. PRG4 has been shown to be present inside the body at the surface of synovium, tendon, articular cartilage such as meniscus, and in the protective film of the eye, among other sites, and plays an important role in joint lubrication and synovial homeostasis.

Lubricin is well known to reduce friction between load bearing surfaces (Swann et al., J Biol Chem, 1981, 256: 5921-5; Schmidt et al., JAMA Ophthalmol, 2013, 131(6):766-76) and therefore research on its potential benefits has been primarily focused on the joint; however, lubricin is expressed in liver, heart, lung, kidney and other tissues (Ikegawa et al., Cytogenet Cell Genet, 2000, 90(3-4):291-7), yet its role in these tissues remains elusive. The disease Camptodactyly Arthropathy Coxa Vara Pericarditis (CACP) is linked to mutations in PRG4 (Marcelino et al., Nat Genet, 1999, 23(3):319-22), and these patients suffer from joint degeneration, chronic inflammation, and pericarditis (Mannurita et al., Eur J Hum Gen, 2014, 22:197-201), which suggests lubricin has multiple biological functions in addition to lubrication.

In humans, lubricin is found in high concentrations in the synovial fluid of the joint (˜400 μg/mL), and has been observed in lower concentrations (>100 μg/mL) in the blood (Ikegawa et al., Cytogenet Cell Genet, 2000, 90(3-4):291-7; Ai et al. PLoS One, 2015, 10(e0116237)). However, as shown herein, Applicant has discovered that lubricin's properties extend beyond its lubricating and anti-adhesive properties. Accordingly, while previous publications show that the loss of lubricin can lead to tissue degeneration (Jay et al., Arthritis Rheum, 2010, 62(8):2382-91; Ruan et al., Transl Med, 2013, 5(176); Jay et al., Matrix Biology, 2014, 39:17-24), Applicant believes it is the first to demonstrate that lubricin plays a key role in wound healing and tissue regeneration. In particular, Applicant has discovered that lubricin is expressed at the site of tissue injury and triggers physiological responses that promote wound healing, thereby suppressing scar formation.

### SUMMARY OF THE INVENTION

The current invention exploits the previously unknown involvement of PRG4 in promoting wound healing and tissue regeneration. Underlying Applicant's discovery is an understanding of putative mechanism through which PRG4 promotes wound healing. As shown herein, PRG4 can enhance endogenous repair and regeneration by 1) inhibiting the fibrotic response, 2) increasing angiogenesis and blood flow to the injury, 3) regulating the inflammatory response (e.g., macrophage polarization) and 4) recruiting immune cells and adult stem cells (MSCs) to mediate tissue repair. Therefore, PRG4 can be used in a number of novel ways to effect wound healing.

Accordingly, in one aspect, the invention provides a method of promoting tissue regeneration or wound healing while reducing scar formation. The method includes administering a pharmaceutical preparation including lubricin to a wound or site of tissue injury in a patient in need thereof. In one embodiment, the pharmaceutical preparation may include a pharmaceutically acceptable carrier.

In one embodiment, the lubricin is recombinant human lubricin. In one embodiment, the lubricin has the amino acid sequence of SEQ ID NO:1. In another embodiment, the lubricin has at least 95% amino acid sequence identity with the amino acid sequence of SEQ ID NO:1. In another embodiment, the lubricin is known homolog or variant of human lubricin.

In one embodiment, the wound is present in the skin. For example, the wound is present in the epidermis, the dermis, and/or the hypodermis of the skin. In one embodiment, the wound is in the epidermis. In another embodiment, the wound is in the epidermis and the dermis. In another embodiment, the wound is in the epidermis, dermis, and hypodermis. In another embodiment, the wound is in the dermis and hypodermis. In another embodiment, the wound is in the hypodermis. In another embodiment, the wound is in the dermis.

In one embodiment, the wound or tissue injury is in the eye. For example, in one embodiment, the wound or tissue injury is in the cornea.

In one embodiment, the wound or tissue injury is a cut, puncture, laceration, tear, burn, scrape or abrasion to the skin, or a surgical incision. In another embodiment, the wound is a bruise. In another embodiment, the wound is a burn. In yet another embodiment, the wound is a dermal ulcer. The ulcer may be a decubitus (pressure) ulcer, a diabetic ulcer or caused by bacterial infection or necrosis. In another embodiment, the wound or tissue injury may be acne. In a further embodiment, the wound or tissue injury is a site from which previously formed scar tissue has been surgically resected. In another embodiment, the wound is at a site that is non-articular, non-osseous, and non-osteal and the wound is not in a bone, joint, articular cartilage, tendon or ligament.

In one embodiment, the lubricin is provided at a concentration of 1 μg/mL to 1 mg/mL. In another embodiment, the lubricin is provided at a concentration of 100 μg/mL. In a further embodiment, the lubricin is provided in an amount of 50 μg to 1000 up per cm² of wound area. In a further embodiment, the lubricin is provided in an amount of 50 μg to 500 μg per cm² of wound area, while in another embodiment, the lubricin is provided in amount of about 75-100 μg per cm² of wound area. In yet another embodiment, the lubricin is provided in an amount of 10-150 μg per cm² of wound area.

In a further embodiment, the pharmaceutical preparation containing lubricin is administered as a solution, suspension, emulsion, lotion, cream, gel, paste, or ointment. In one embodiment, the solution is administered as drops topically at the site of tissue injury or by injection at the site of tissue injury. In one embodiment, the pharmaceutical preparation is applied topically at the site of tissue injury, or is administered by injection to the site of injury. In a further embodiment, the pharmaceutical preparation does not include hyaluronic acid.

In another embodiment, the pharmaceutical preparation is administered to the wound or tissue injury as an impregnate of a wound dressing applied to the wound.

In one embodiment, the pharmaceutical preparation is administered with an analgesic or the administered pharmaceutical composition contains an analgesic. For example, the analgesic is lidocaine or benzocaine.

In one embodiment, the pharmaceutical preparation is administered with an antibiotic or anti-inflammatory agent, or the administered pharmaceutical composition contains and antibiotic or an anti-inflammatory agent. For example, the antibiotic is neomycin, polymyxin b, bacitracin, erythromycin, retapamulin, sulfacetamide sodium, mupirocin, pramoxine, silver sulfadiazine, mafenide, or ozenoaxacin. For example, the anti-inflammatory agent is hydrocortisone.

In one embodiment, the invention provides a pharmaceutical composition including lubricin for use in promoting tissue regeneration or wound healing while reducing scar formation. In one embodiment, the pharmaceutical composition may include a pharmaceutically acceptable carrier.

In another embodiment, the invention provides a method of inducing angiogenesis at a site in a body of a patient in need thereof. The method involves administering a pharmaceutical preparation including lubricin to the site in an amount sufficient to induce new blood vessel formation. In one embodiment, the pharmaceutical preparation may include a pharmaceutically acceptable carrier.

In one embodiment, the lubricin is recombinant human lubricin. In one embodiment, the lubricin has the amino acid sequence of SEQ ID NO:1. In one embodiment, the lubricin has at least 95% amino acid sequence identity with the sequence of claim 1. The lubricin may be provided at a concentration of 1 μg/mL to 1 μg/mL, for example, at 100 μg/mL. The lubricin may be provided in an amount of 10-150 μg per cm² area of the site, while in another embodiment, the lubricin is provided in the amount of 75-100 μg per cm² area of the site.

In order to promote angiogenesis, the lubricin may administered topically to the site, or to the site by injection, for example. In one embodiment, the pharmaceutical preparation does not include hyaluronic acid. In a further embodiment, the site in need of angiogenesis is the site of a tissue injury or wound such as a cut, puncture, laceration, tear, scrape, or abrasion to the skin, a surgical incision, or a burn or bruise or ulcer. For example, the site of the tissue injury or wound may be the epidermis and may include the dermis or hypodermis depending on the depth of the wound.

In one embodiment, the site of the tissue injury or wound is in the eye, but not in the cornea.

In a further embodiment, the invention provides a pharmaceutical composition for use in promoting angiogenesis that includes lubricin. In one embodiment, the pharmaceutical composition may include a pharmaceutically acceptable carrier.

In one aspect the invention provides a method of treating or preventing aberrant wound healing in an eye. The method involves administering lubricin to an eye suffering from or at risk of aberrant wound healing. In one embodiment, the aberrant wound healing is scarring of the cornea, while in another embodiment it is corneal haze. According to one embodiment, the lubricin is provided at a concentration of 1 μg/mL to 1 mg/mL and/or in an amount of 10-150 μg per cm² based on the size of the wound. In one embodiment, the lubricin has at least 95% amino acid sequence identity with the sequence of SEQ ID NO:1 or residues 25-1404 of SEQ ID NO:1. In one embodiment, the lubricin is administered ophthalmically as drops or an ointment. In one embodiment, the lubricin is administered to the eye upon completion of a keratinoplasty, photorefractive keratectomy, laser subepithelial keratomileusis, or laser in situ keratomileusis.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic diagram showing a proposed mechanism by which lubricin regulates wound healing.

FIG. 2 is a line graph showing the diameter (mm) of a wound in the ear of C57BL/6 mice (initial 2 mm diameter punch) as measured over the course of weeks from the time of initial injury. It shows that lubricin treatment significantly increases wound healing, while knocking out lubricin blocks wound healing (n=12/group).

FIG. 3 includes four photographs of immunohistochemical staining of tissue from mouse ears. The green stain shows the presence of lubricin, whereas blue stain shows the presence of cells not expressing lubricin. As shown, in the lubricin knock out mouse, no lubricin is present (bottom left panel) as represented by the presence of blue staining only, whereas in the MRL “super healer” mice, lubricin is abundant throughout the sample (top right panel) as represented by green staining only throughout the tissue. In the C57BL/6 lubricin treated mice, lubricin is visible throughout the tissue (bottom right panel), as represented by green staining throughout although not to the same extent as in the MRL mice, whereas in untreated C57BL/6 mice, no lubricin is present (top left panel) as represented by blue staining throughout with only a few minor spots of green. Accordingly, FIG. 3 shows that lubricin staining is increased after injury in spontaneous super-healer (e.g. MRL mice) and lubricin staining is correlated with increased healing and decreased fibrosis/scaring after injury.

FIG. 4 is a line graph showing blood flow in perfusion units (PU) in mice ears at the site of the punch injury as measured over the course of weeks from the time of initial injury and shows that lubricin treatment significantly increases blood flow at the injury site (n=12/group).

FIG. 5 is a line graph showing the level of angiogenesis in mice ears suffering from a punch injury, as demonstrated by the number of CD31+ vessels in the healing tissue surrounding the injury site. After ear injury, increased numbers of blood vessels are observed in lubricin treated animals, while fewer vessels are observed in lubricin KO animals (n=12).

FIG. 6 is a series of images of immunohistochemical staining of mice ear tissue with DAPI, or 4′,6-diamidino-2-phenylindole, where the tissue has not been injured (top panel), has been injured and a carrier was administered (middle panel), and has been injured and PRG4 was administered (bottom panel). Red indicates the presence of mesenchymal stem cells (MSCs), of which none are present in non-injured tissue, a few are present in injured tissue administered carrier alone, and many are present in the injured tissue administered lubricin as indicated by red dots throughout the image. FIG. 6 shows that MSCs migrate to areas of injury and respond to lubricin. In the ear, MSCs (red) are not observed in quantity in uninjured tissue, but can be observed after injury. With lubricin treatment, MSCs completely repopulate the damaged cartilaginous structure of the ear (n=5/group).

FIG. 7 is a line graph of demonstrating PAI-1 (Plasminogen activator inhibitor-1) lubricin binding affinity as measured in resonance units (RU) versus concentration as measured by surface plasmon resonance. Lubricin is able to bind directly PAI-1 and the binding constant was 2.712×10⁻⁶M, indicating a strong binding between the two biomolecules.

FIGS. 8A-E are graphs showing how lubricin up-regulates HIF1a and VEGF in multiple cell types and in vivo. Synovial fibroblasts from normal and osteoarthritic (OA) human joints were exposed to lubricin (100 μg/mL) (FIGS. 8A and 8B). In normal and OA cells, lubricin significantly up-regulated expression of HIF1a and VEGF mRNA. This observation was validated in HEK293 cells, where it was observed that HIF1a and VEGF mRNA were both up-regulated after lubricin treatment (FIG. 8C). In the bar graphs of FIGS. 8A-C, lubricin treatment is shown as the bar on the left (topped by an *) and PBS control is the bar on the right, and each of FIGS. 8A-C, the lubricin bar is taller than the PBS bar. ELISA was used to validate the levels of VEGF (FIG. 8D). In FIG. 8D, from left to right, the two left-most bars represent protein concentration in pg/mL in normal synovial cells treated with PBS (left) or lubricin (right), the middle two bars represent protein concentration in pg/mL in osteoarthritic synovial cells treated with PBS (left) or Lubricin (right) and the right-most bars represent protein concentration in pg/mL in HEK293 cells treated with PBS (left) or lubricin (right). FIG. 8E shows that in rats injected with lubricin, VEGF was up-regulated systematically 14-28 days after lubricin injection. The top line having of the bar graph with points at Day 14, 29, and 24 having * present represents VEGF levels in rats treated with lubricin, whereas the lower line represents VEGF levels in untreated rats.

FIG. 9 is a bar graph showing levels of IL-1, IL-6, and TNFα secreted from macrophages in C57BL/6 versus lubricin knock out mice (KO). (* indicates a significance of p<0.05). Polarization of lubricin KO macrophages leads to an increase of pro-inflammatory factors, compared to wild-type controls (n=4).

FIG. 10 is a bar graph showing levels of HIF1a and VEGF in TLR4 knock out cells, TLR4 knock out cells administered lubricin, lubricin knock out cells, and lubricin knock out cells administered PAI-1. Levels of gene expression are normalized to the ribosome subunit 18s. HIF1α and VEGF up-regulation after lubricin treatment is independent of TLR4 and PAI-1 (n=5).

FIG. 11 is flow cytometric data showing the presence of CD14+ macrophages and GR1+ neutrophils in C57BL/6 mice, C57BL/6 mice administered lubricin, and in lubricin knock out (KO) mice at the injury site one week after ear injury. As the data shows, after injury, twice as many neutrophils and macrophages are found within the injury site with lubricin treatment as compared to the C57BL/6 and lubricin KO mice. (n=6 per group with 3 males and 3 females).

FIG. 12 is the amino acid sequence of full length (non-truncated) human PRG4 (SEQ ID NO:1: 1404 residues). Residues 1-24 (shown in bold) represent the signal sequence and residues 25-1404 represent the mature sequence of human PRG4. The glycoprotein does not require the signal sequence in its active form.

FIGS. 13A-C provide the nucleic acid sequence for the PRG4 gene (SEQ ID NO:2) encoding the full length 1404 AA human PRG4 protein.

FIG. 14A is a photograph of ear punch wounds in a C57BL/6 mouse and in a PRG4 knockout mouse 4 weeks after injury. The original injury was a 4 mm² punch. The left ear was treated with carrier (DMSO) as a control, whereas the right ear was treated with rhPRG4. The punch wounds in the PRG4 treated ears have closed significantly compared to the untreated ears.

FIG. 14B is a line graph of wound area in mm² vs time after injury in weeks for C57BL/6 mice and PRG4−/− mice receiving ear punch injuries and treated with DMSO or rhPRG4 as indicated. The dotted lines represent PRG4 treatment. N=12 per group with 6 males and 6 females. The original injury was a 4 mm² punch.

FIGS. 14C-D are line graphs of wound area in mm² vs time after injury in weeks for C57BL/6 mice, PAI−/− mice, TLR4−/− mice and PAI−/− TLR4−/− mice receiving ear punch injuries and treated with DMSO or rhPRG4 as indicated. N=12 per group with 6 males and 6 females. The original injury was a 4 mm² punch.

FIG. 15 is a series of bar graphs showing the relative expression of VEGF (top), TGFI3 (middle) and PRG4 (bottom) in macrophages (F4/80+; left) and mesenchymal stem cells (MSCs; right) on a given day since injury with an ear punch. (n=3 per timepoint).

FIGS. 16A-B include flow cytometric data showing the presence of CD38+ macrophages (M1) and CD206+ macrophages (M2) in C57BL/6 mice, PRG4−/− knock out mice and TLR4−/− as well as bar graphs showing the percentage of CD38+ cells in the various treatment groups.

FIG. 17 shows a series of images of immunohistochemical staining of mice ear tissue with αSMA (white) 4 weeks post injury. The white line depicts the original wound site. (n=6 per group with 3 males and 3 females).

FIGS. 18A-B include flow cytometric data showing the presence of macrophages (F4/80+) and MSCs (Sca1+CA140a+) taken from non-injured C57BL/6 mice and TLR4−/− treated with rhPRG4 or DMSO as indicated as well as bar graphs showing the relative NFκB expression or relative gene expression in the various treatment groups as shown. (n=6 per group with 3 males and 3 females).

FIG. 19 is a line graph showing blood flow at the site of injury in mice ears relative to uninjured blood flow levels prior to injury measured over a period of weeks from injury. rhPRG4 treatment significantly increased blood flow at the injury site. (n=6 per group with 3 males and 3 females). The original injury was a 4 mm² punch.

FIG. 20 is a scatter plot of the number of CD31+ vessels per mm² within an injured mouse ear one week after injury in various treatment groups as shown. (n=6 per group with 3 males and 3 females). The original injury was a 4 mm² punch.

FIG. 21 is a series of images of immunohistochemical staining of mice ear tissue with DAPI, or 4′,6-diamidino-2-phenylindole. In the top panels, the presence of blue staining is prevalent throughout the tissue sample is indicative of nuclei, and presence of red staining, also present throughout the image is indicative of MSCs The contribution of these MSCs is traced in the bottom left panel (treatment with PRG4) to new tissues (#1 skin, #2 cartilage, and #3 hair follicles), whereas in the DMSO treated mice on the right panel, the MSCs contributed to fibrotic-like tissue (#4).

### DETAILED DESCRIPTION OF THE INVENTION

Wound healing is a complex process by which tissues repair themselves after injury and involves various physiological processes including hemostasis, inflammation, proliferation and growth of new tissue including angiogenesis, and remodeling or maturation of tissue. Wounds are injuries involving an external or internal break in body tissue. For example, wounds can be internal, involving breakage of the epithelial layer of an organ or tissue inside the body or they can be external, involving breakage of the skin or epithelial layer of the eye. Wound healing refers to the process by which such wounds are repaired and the break in body tissue closes.

The wound healing response is one of most primitive and conserved physiological responses in the animal kingdom, as restoring tissue integrity/homeostasis can be the difference between life and death. Wound healing in mammals is mediated primarily by immune cells and inflammatory signaling molecules that can regulate other tissue resident cells, including adult stem cells, to mediate closure of the wound through formation of a scar. As demonstrated herein, Applicant has observed that lubricin, a protein found throughout the animal kingdom from fish to elephants (Ikegawa et al., Cytogenet Cell Genet, 2000, 90(3-4):291-7), is highly expressed in stem cells immediately after injury.

As demonstrated herein, lubricin enhances endogenous wound repair and tissue regeneration by 1) inhibiting the fibrotic response, 2) increasing angiogenesis and blood flow to the injury, 3) regulating the inflammatory response (e.g. macrophage polarization toward M2 vs M1) and 4) recruiting immune cells and adult stem cells (MSCs) to mediate tissue repair.

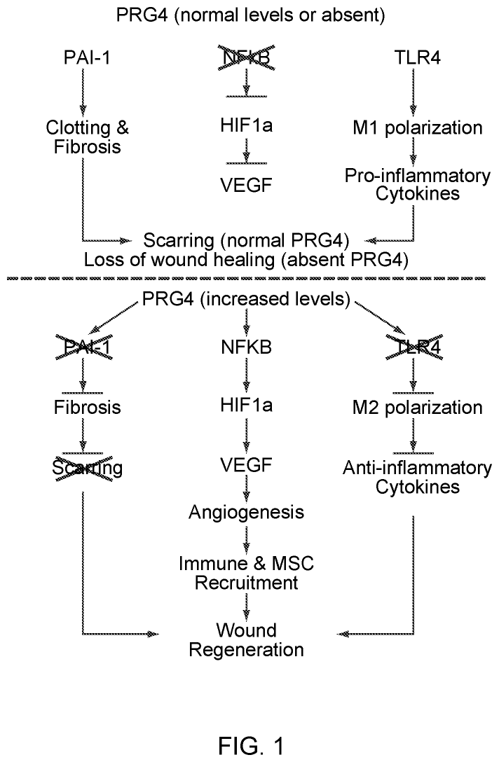

FIG. 1 diagrams the potential mechanism by which lubricin achieves these effects. Essentially, when normal levels of lubricin are present at a wound site, fibrosis and increased pro-inflammatory cytokines lead to scarring, whereas when lubricin is present at the wound site at increased levels over what is physiologically available, fibrosis is inhibited, macrophages exhibit M2 polarization leading to expression of anti-inflammatory cytokines, and other pathways are activated leading to angiogenesis and immune and mesenchymal stem cell (MSC) recruitment, resulting in superior wound healing with minimal to no scar formation. Accordingly, according to the invention and as demonstrated herein, lubricin promotes wound healing and tissue regeneration by not only reducing or inhibiting fibrosis which causes scar formation, but also by promoting macrophages to exhibit M2 polarization leading to expression of anti-inflammatory cytokines, and by activation of other pathways leading to angiogenesis and immune and mesenchymal stem cell (MSC) recruitment.

While many tissues demonstrate adequate wound healing under normal circumstances, in cases where the primary insult (injury/disease) persists, this can result in chronic inflammation and inappropriate fibrotic repair. Chronic inflammation prevents healing, as evident in ulcerative lesions. While continued fibrosis in the skin leads to scarring and disfigurement, progressive deposition of matrix in organs compromises their structure and function, causing disease and death (Gurtner et al., Nature, 2008, 453:314).

Understanding the pathways regulated by lubricin in wound healing described herein suggests that lubricin may be used to promote regeneration of injured tissue by normal cells of the same kind and that by regulating the expression of lubricin, wound healing can be enhanced by triggering a regenerative response in place of the fibrotic repair response.

The fibrotic repair response results in scar tissue formation. In this physiological process, injured tissue is replaced with scar tissue (rather than normal tissue) due to fibrosis, resulting in the deposition of collagen in a manner different than in normal skin. However, the methods of the invention promote a physiological approach to wound healing that results in regeneration or neogenesis of tissue, that is, replacement of injured tissue with new, normal and functional tissue, rather than fibrous scarring deposits. For example, according to methods of the invention, damaged or injured tissue is replaced with normal, healthy cells/tissue, and scar formation is reduced or eliminated at the site of injury. As demonstrated herein, this regenerative response is due lubricin's ability to promote angiogenesis, inhibit the fibrotic response, promote macrophage polarization toward M2 from M1 thereby modulating the inflammatory response from pro-inflammatory to anti-inflammatory and to recruit immune cells and adult stem cells (MSCs) to mediate tissue repair and the site of tissue injury.

Accordingly, the invention provides a method of promoting wound healing or tissue regeneration while reducing scar formation wherein lubricin (PRG4) is administered to the wound. According to one embodiment of the invention, the level of tissue regeneration, observed at the site of the wound is greater than what would occur without the use of exogenous lubricin, and the level of scar formation is reduced compared to what would occur without the use of exogenous lubricin.

### PRG4 Protein

PRG4, also referred to as lubricin, is a lubricating polypeptide, which in humans is expressed from the megakaryocyte stimulating factor (MSF) gene, also known as PRG4 (see NCBI Accession Number AK131434-U70136). Lubricin is a ubiquitous, endogenous glycoprotein that coats the articulating surfaces of the body. Lubricin is highly surface active molecule (e.g., holds onto water), that acts primarily as a potent cytoprotective, anti-adhesive and boundary lubricant. The molecule has a long, central mucin-like domain located between terminal protein domains that allow the molecule to adhere and protect tissue surfaces. Its natural form, in all mammals investigated, contains multiple repeats of an amino acid sequence which is at least 50% identical to KEPAPTT (SEQ ID N0:3). Natural lubricin typically comprises multiple redundant forms of this repeat, which typically includes proline and threonine residues, with at least one threonine being glycosylated in most repeats. The threonine anchored O-linked sugar side chains are critical for lubricin's boundary lubricating function. The side chain moiety typically is a β(1-3)Gal-GalNAc moiety, with the β(1-3)Gal-GalNAc typically capped with sialic acid or N-acetylneuraminic acid. The polypeptide also contains N-linked oligosaccharides. The gene encoding naturally-occurring full length lubricin contains 12 exons, and the naturally-occurring MSF gene product contains 1,404 amino acids (including the secretion sequence) with multiple polypeptide sequence homologies to vitronectin including hemopexin-like and somatomedin-like regions. Centrally-located exon 6 contains 940 residues. Exon 6 encodes the repeat rich, O-glycosylated mucin-like domain.

The amino acid sequence of the protein backbone of lubricin may differ depending on alternative splicing of exons of the human MSF gene. This robustness against heterogeneity was exemplified when researchers created a recombinant form of lubricin missing 474 amino acids from the central mucin domain, yet still achieved reasonable, although muted, lubrication (Flannery et al., Arthritis Rheum 2009; 60(3):840-7). PRG4 has been shown to exist not only as a monomer but also as a dimer and multimer disulfide-bonded through the conserved cysteine-rich domains at both N- and C-termini Lubris, LLC has developed a full-length recombinant form of human lubricin. The molecule is expressed using the Selexis Chinese hamster ovary cell line (CHO-M), with a final apparent molecular weight of 450-600 kDa, with polydisperse multimers frequently measuring at 1,000 kDa or more, all as estimated by comparison to molecular weight standards on SDS tris-acetate 3-8% polyacrylamide gels. Of the total glycosylations, about half comprise two sugar units (GalNAc-Gal), and half three sugar units (GalNAc-Gal-Sialic acid). This method of recombinant human PRG4 production is disclosed in International Patent Application No. PCT/US014/061827.

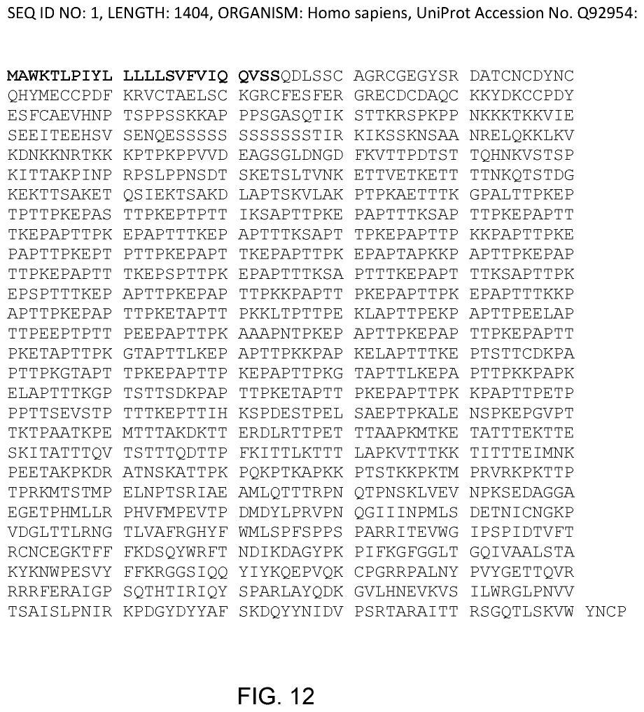

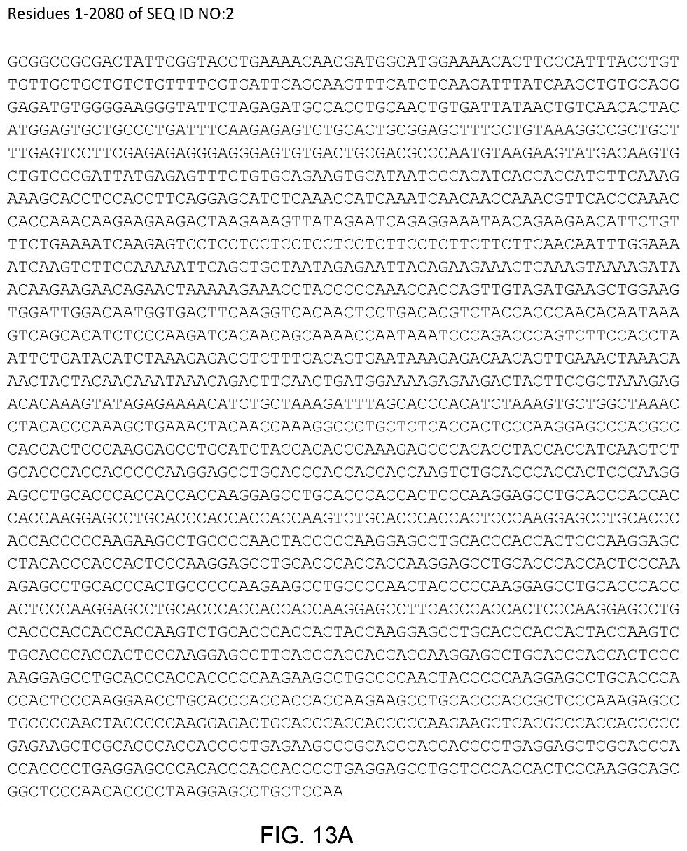

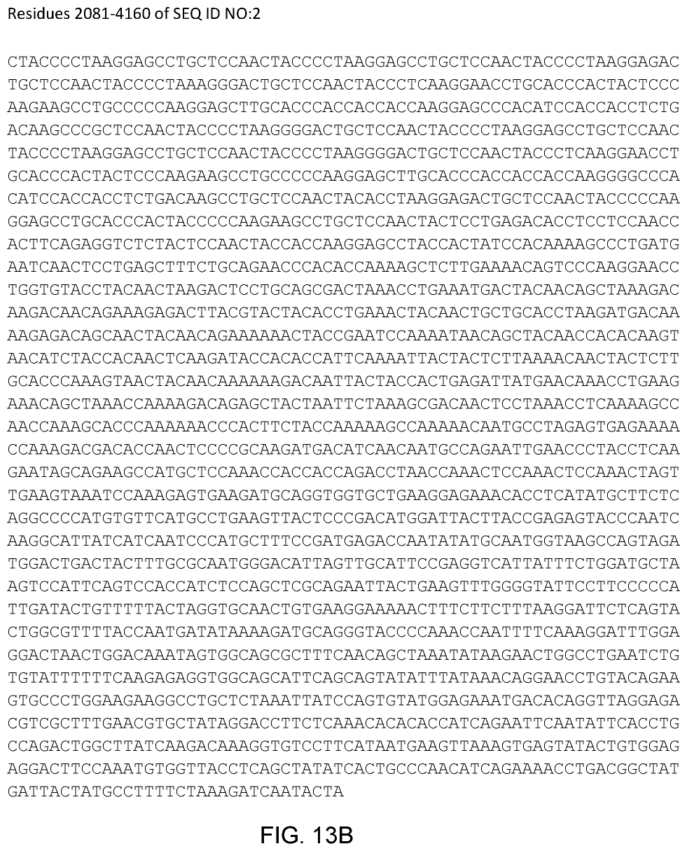

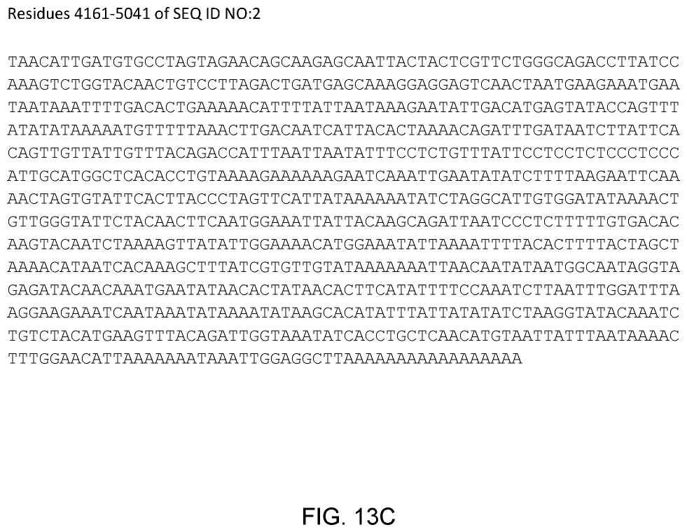

Any one or more of various native and recombinant PRG4 proteins and isoforms may be utilized in the various embodiments described herein. For instance, U.S. Pat. Nos. 6,433,142; 6,743,774; 6,960,562; 7,030,223, and 7,361,738 disclose how to make various forms of human PRG4 expression product, each of which is incorporated herein by reference. Preferred for use in the practice of the invention is full length, glycosylated, recombinant PRG4, or lubricin, expressed from CHO cells. This protein comprises 1,404 amino acids (see FIG. 12; SEQ ID NO:1) including a central exon comprising repeats of the sequence KEPAPTT (SEQ ID NO: 3) variously glycosylated with O-linked β(1-3) Gal-GalNAc oligosaccharides, and including N and C-terminal sequences with homology to vitronectin. The molecule is polydisperse with the glycosylation pattern of individual molecules varying, and can comprise monomeric, dimeric, and multimeric species.

As used herein, the term “PRG4” is used interchangeably with the term “lubricin.” Broadly, these terms refer to any functional isolated or purified native or recombinant PRG4 proteins, homologs, functional fragments, isoforms, and/or mutants thereof. All useful molecules comprise the sequence encoded by exon 6, or homologs or truncated versions thereof, for example, versions with fewer repeats within this central mucin-like KEPAPTT-repeat domain, preferably together with 0-linked glycosylation. All useful molecules also comprise at least the biological active portions of the sequences encoded by exons 1-5 and 7-12, i.e., sequences responsible for imparting to the molecule its affinity for ECM and endothelial surfaces. In certain embodiments, a preferred PRG4 protein has an average molar mass of between 50 kDa and 500 kDa, preferably between 224 to 467 kDa, comprising one or more biological active portions of the PRG4 protein, or functional fragments, such as a lubricating fragment, or a homolog thereof. In a more preferred embodiment, a PRG4 protein comprises monomers of average molar mass of between 220 kDa to about 280 kDa.

In some embodiments, functional or biologically active PRG4 fragments and homologs are contemplated that have at least 50%, 55%, 60%, 65%, 70%, 75%, 80%, 85%, 90%, 95%, or 98% amino acid sequence identity with SEQ ID NO:1 or with the sequences encoded by exons 1-5 and 7-12 of PRG4, or with residues 25-1404 of SEQ ID NO:1. In some embodiments, functional or biologically active PRG4 fragments and homologs are contemplated that have at least 50%, 55%, 60%, 65%, 70%, 75%, 80%, 85%, 90%, 95%, or 98% sequence identity with residues 25-1404 of SEQ ID NO:1. In another embodiment, the PRG4 is recombinant human lubricin. In another embodiment, the PRG4 has the amino acid sequence of SEQ ID NO:1. In another embodiment, the PRG4 has the amino acid sequence of residues 25-1404 SEQ ID NO:1.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino acid or nucleic acid sequence). The percent identity between the two sequences is a function of the number of identical positions shared by the sequences (i.e., % homology=(# of identical positions/total # of positions)times 100). The determination of percent homology between two sequences can be accomplished using a mathematical algorithm. A non-limiting example of a mathematical algorithm utilized for the comparison of two sequences is the algorithm of Karlin and Altschul, (1990) Proc. Natl. Acad. Sci. USA, 87:2264-68, modified as in Karlin and Altschul, (1993) Proc. Natl. Acad. Sci. USA, 90:5873-77. Such an algorithm is incorporated into the NBLAST and XBLAST programs of Altschul et al., (1990) J. Mol. Biol., 215:403-10. BLAST nucleotide searches can be performed with the NBLAST program, score=100, wordlength=12. BLAST protein searches can be performed with the XBLAST program, score=50, wordlength=3. To obtain gapped alignments for comparison purposes, Gapped BLAST can be utilized as described in Altschul et al., (1997) Nucleic Acids Research, 25(17):3389-3402. When utilizing BLAST and Gapped BLAST programs, the default parameters of the respective programs (e.g., XBLAST and NBLAST) can be used.

In some embodiments, functional PRG4 fragments and homologs are contemplated that have at least 50%, 55%, 60%, 65%, 70%, 75%, 80%, 85%, 90%, 95%, or 98% activity as compared with native PRG4, e.g., biological activity.

Methods for isolation, purification, and recombinant expression of a proteins such as PRG4 protein are well known in the art. In certain embodiments, the method starts with cloning and isolating mRNA and cDNA encoding PRG4 proteins or isoforms using standard molecular biology techniques, such as PCR or RT-PCR. The isolated cDNA encoding the PRG4 protein or isoform is then cloned into an expression vector and expressed in a host cell for producing recombinant PRG4 protein, and isolated from the cell culture supernatant. A method for production of recombinant human PRG4 is provided in International Patent Application No. PCT/US014/061827.

### Types of Wounds or Tissue Injury to be Treated

As previously stated, wounds are injuries involving an external or internal break in body tissue. For example, wounds can be internal, involving breakage of the epithelial layer of an organ or tissue inside the body or they can be external, involving breakage of the skin or epithelial layer of the eye. Wound healing refers to the process by which such wounds are repaired and the break in body tissue closes.

In one aspect, the methods of the invention provide for treatment of wounds or injured tissue of the skin with PRG4. Wounds may be in the epidermis, dermis, or hypodermis, or in one or more of these layers. Tissue injury may be below the surface of the skin, such as is the case with bruising, hematoma, or necrosis when the epidermis may not yet be compromised, but damage to the dermis, hypodermis and even muscle tissue is present.

The methods of the invention contemplate treatment of wounds to the skin such as cuts, lacerations, tears, burns, bruises, abrasions, punctures, or surgical incisions. Treatment of wounds such as ulcers are also contemplated by the invention. For example, pressure ulcers (bed sores or decubitus), diabetic ulcers, or ulcers caused by necrosis or infection may be treated with lubricin according to the methods of invention to promote tissue regeneration and reduce scar formation. Treatment of acne is also contemplated, as acne involves breaking of the epithelial tissues of the skin.

The methods of the invention also contemplate treatment of wounds to the eye and treatment of aberrant wound healing in the eye using PRG4. Aberrant wound healing and wound healing disorders in the eye may lead to severe ocular tissue damage via activation of inflammatory cells, release of growth factors and cytokines, proliferation and differentiation of ocular cells, increased capillary permeability, alterations in basement membrane matrix compositions, increased depositions of extracellular matrix, fibrosis, neovascularization and tissue remodeling. In particular, aberrant wound healing in the cornea may result in production of blood and lymphatic vessels which are not present in healthy cornea. Further, because injury to the corneal stroma does not always induce corneal scarring, corneal scarring is the result of aberrant wound healing in the eye.

Accordingly, methods of the invention provide for treatment of wounds or injured tissue of the eye using PRG4. In one embodiment, a method of the invention provides for the treatment of aberrant wound healing or a wound healing disorder in the eye by administering lubricin to the eye. The lubricin may be administered to a specific location of a wound in the eye or it may be administered generally to eye. Some wounds that may be treated include those caused by ocular surgery to the eye. For example, in one embodiment, the methods of invention prevent and/or treat corneal haze resulting from exposure of the eye to laser irradiation. In one embodiment, the methods of the invention promote wound healing in the cornea, thereby preventing or inhibiting the formation of corneal scar tissue associated with surgical insults or trauma to the cornea, including trauma associated with invasive or non-invasive corneal surgery. For example, trauma to the cornea may result from a laser keratoplasty procedure or photorefractive keratectomy (PRK) or laser in situ keratomileusis (LASIK) or laser subepithelial keratomileusis (LASEK). In other embodiments, administration of PRG4 to the eye promotes corneal wound healing. For example, administration of PRG4 to the eye according to the methods of the invention can be used to treat or prevent corneal scarring. Corneal scarring may be caused by, for example, shingles, herpes simplex 1 or 2, contact lens use, or scratching or burning of the cornea due accident or injury, among other things. In some embodiments, PRG4 is administered to an eye to reduce the presence of pre-existing scar tissue, for example, in the cornea, which can cause blurred or reduced vision, due to clouding of the cornea. According to one embodiment, the eye is a human eye. According to one embodiment, the eye is at risk of aberrant wound healing when the eye is subject to disease or injury. For example, surgical insult, such as but not limited to PRK, LASEK, or LASIK or accidental injury or the eye from objects touching the eye can place the eye at risk for aberrant wound healing. For example, disease, such as aforementioned viral illness that can damage the eye also places the eye at risk of aberrant wound healing.

The methods of the invention provide for treatment of internal injuries to tissue and organs beyond the skin, for example, as the result of trauma such as from falls, car accidents, stabbing or knife wounds, gun shots, or blunt force trauma; internal bleeding or bruising; or from surgical insults. Accordingly, lubricin can be administered to tissues of the nervous system (brain, spinal cord, nerves), muscle tissue, epithelial tissue, or connective tissue to promote wound healing and tissue regeneration while reducing the formation of scar tissue. Treatment of wounds and reduction of scaring to the ears, eyes, lips, kidney, liver, pancreas, lung, stomach, esophagus, trachea, bladder, intestine, colon, heart, endocrine organs, or blood vessels are also contemplated by the invention. For treatment of internal injuries, lubricin may be administered by local injection, or topically via surgical intervention. Intravenous systemic administration for wound healing is also contemplated. The invention contemplates treatment of surgical wounds in the body, such as those that perforate or breach epithelial tissues such as in the gastrointestinal tract.

In one embodiment, the wound to be treated is in a location that is non-osseous, non-osteal, and non-articular. In one embodiment, the wound to be treated is in not a wound in a bone or a joint or articular cartilage or a tendon or ligament. Accordingly, the invention includes methods of promoting tissue regeneration or wound healing while reducing scar formation where PRG4 is administering to a wound or the site of tissue injury in a patient in need thereof where the wound is not in a bone or a joint or articular cartilage or a tendon or ligament. The invention includes methods of promoting tissue regeneration or wound healing while reducing scar formation where PRG4 is administering to a wound or the site of tissue injury in a patient in need thereof where the wound is in a location that is non-osseous, non-osteal, and non-articular. In one embodiment, the wound to be treated is in not a wound in cartilage. Accordingly, the invention includes methods of promoting tissue regeneration or wound healing while reducing scar formation where PRG4 is administering to a wound or the site of tissue injury in a patient in need thereof where the wound is not in cartilage. In one embodiment, the wound to be treated is a wound in cartilage that is not articular cartilage. Accordingly, the invention includes methods of promoting tissue regeneration or wound healing while reducing scar formation where PRG4 is administering to a wound or the site of tissue injury in a patient in need thereof where the wound is not articular cartilage. For example, the cartilage could be in the ear or the nose.

The invention also provides methods for removing or reducing the appearance of existing scars in a patient, whether on the skin or elsewhere in the body. According to such methods, scar tissue may be removed, for example, by surgical resection, and lubricin provided to the area of scar removal to promote tissue regeneration at the site of the previously existing scar.

The methods of invention provide that a pharmaceutical composition containing recombinant human lubricin may be applied to a wound or tissue injury as an impregnate of a wound dressing. The wound dressing may be a gauze, pad, or bandage. Materials that may be impregnated with lubricin include cotton, polyester, rayon, or blends of the aforementioned fabrics, calcium or sodium alginate, polyethylene, polyurethane film or foam, polyacrylate, polypropylene, cellulose, polyester film, nylon, elastane, or other suitable material for a bandage or wound dressing.

According to methods of the invention, the wounds or tissue injury to be treated are on/in a human patient. However, treatment of horses or dogs, or other mammals is also contemplated. In such veterinary applications, native or recombinant lubricin from the particular species being treated may be used.

Further, the invention also provides methods for inducing angiogenesis. For example, according to the invention, a method of inducing angiogenesis involves applying a pharmaceutical composition that includes lubricin to a site on a patient's body that is in need of angiogenesis. The site could be, for example, a wound, such as a cut, burn, abrasion, puncture, or laceration, of the skin, or the site could be an injury in an organ or other tissue of a human body.

### Administration of PRG4

While PRG4 is produced naturally within the body, the effects of the invention are observed when exogenous PRG4 is administered to a site of tissue injury in the patient. Accordingly, in one embodiment, the PRG4 administered to the patient is exogenous human PRG4, while in another embodiment, the PRG4 administered to the patient is recombinant human PRG4 (rhPRG4). In another embodiment, rhPRG4 has the sequence of SEQ ID NO:1 while in another embodiment, the rhPRG4 has the sequence of residues 25-1404 of SEQ ID NO:1.

PRG4 can be administered to the patient by a number of methods. For example, the PRG4 may be administered topically to the site of a wound, or the PRG4 may be administered locally to the site of a wound, for example, by injection. PRG4 may also be administered systemically by intravenous administration.

The amount of PRG4 administered will depend on variables such as the size of the wound, the depth of the wound, and the location of the wound in the body.

In some embodiments, a therapeutically effective amount of PRG4 for administration to a site of tissue injury either topically or by local injection according to the invention is in the range of 0.1 μg/kg to 4000 μg/kg, or 0.1 μg/kg to 1000 μg/kg, or 0.1 μg/kg to 100 μg/kg, or 0.1 to 50 μg/kg. In some embodiments, the therapeutically effective amount of PRG4 administered is in the range of 0.1 mg/kg to 100 mg/kg, or 1 mg/kg to 100 mg/kg, or 1 mg/kg to 10 mg/kg.

The PRG4 administered may also be in a range of 0.1 μg/mL to 30 mg/mL, or 1 μg/mL to 10 mg/mL, or 10 μg/mL to 1 mg/mL, or 10 μg/mL to 500 μg/mL, or 50 μg/mL to 150 μg/mL In some embodiments, PRG4 is administered at concentrations of about 100 μg/mL. In some embodiments, PRG4 is administered in small volumes of 1 to 100 μL per dose.

In certain embodiments, a total amount of 2 mg to 10 mg of lubricin is administered to the wound at a time, e.g., 2 mg to 10 mg, 2 mg to 5 mg, 2 mg to 3 mg, 3 mg to 4 mg, 4 mg to 5 mg, 5 mg to 6 mg, 6 mg to 7 mg, 7 mg to 8 mg, 8 mg to 9 mg, 9 mg to 10 mg, or 5 mg to 10 mg. In certain embodiments, more than 10 mg of lubricin is administered to the wound. It is contemplated in this invention that the dose of PRG4 used for intravenous administration is at least 1.5 fold, or at least 2 fold, or at least 3 fold, or at least 4 fold, or at least 5 fold, or at least 10 fold higher than dose used for topical or local administration.

In certain embodiments, the amount of lubricin administered to the wound is based on the size of the wound. For example, the amount of lubricin administered is in the range of 10 μg to 1000 μg per cm² of wound area, or in the range of 50 μg to 500 μg per cm² of wound area, or in the range of 50 μg to 300 μg per cm² of wound area, or in a range of 10 μg to 150 μg per cm² of wound area, or in the range of 5 μg to 100 μg per cm² of wound area, or in the range of 25 μg to 150 μg per cm² of wound area, or in the range of 40 μg to 120 μg per cm² of wound area, or in the range of 70 μg to 100 μg per cm² of wound area, or in the range of 50 μg to 150 μg per cm² of wound area in the range of 75 μg to 100 μg per cm² of wound area, or in the amount of about 80 μg per cm² of wound area. Depending on the formulation of the pharmaceutical composition for delivering the lubricin, the amount of lubricin in the formulation may need to be adjusted to ensure delivery of the aforementioned amounts of lubricin from the carrier to the wound site. Adjustment of the formulation to ensure delivery from the carrier of therapeutically effective quantities of lubricin is within the skill in the art.

Administration of lubricin to the site of tissue injury may be carried out every day, every other day, every three days, every four days, every five days, every six days, once weekly, or every other week until wound healing is complete. A complete treatment may take for example, one week, two weeks, three weeks, or one month. In the case of treating chronic skin conditions, treatment may continue for as long as the condition requires for resolution.

Systemic administration of PRG4 is also contemplated by some embodiments of the invention. For example, PRG4 may be systemically administered in an enteral manner, such as oral, rectal, sublingual, sublabial, or buccal delivery. PRG4 may be systemically administered in a parenteral manner, such as nasal, by inhalation, intravenous, intramuscular, subcutaneous, intradermal, or transmucosal delivery.

For delivery of PRG4 to the eye, lubricin may be incorporated into a solution, suspension, emulsion or gel. Ophthalmic compositions of PRG4 can be delivered to the eye via, for example, the use of a collagen shield, contact lenses, or other solid matrices capable of delivering drugs to the cornea placed on the ocular surface. PRG4 may also be delivered to the eye as drops or ointment. For example, PRG4 may be applied to the eye's surface or to the cul de sac of the eye, or by irrigation of the eye. In ophthalmic applications, PRG4 should be delivered to the location of a wound or injury as soon as possible after the wound or injury occurs and via a method that allows the PRG4 to stay in contact with the site of wound or injury and not be quickly flushed away into the tear ducts. However, if this does occur, reapplication of the PRG4 may be necessary to ensure continued contact with the wound surface to ensure healing.

A preferred route of systemic administration of PRG4 contemplated herein is intravenous administration. The optimal dose can be determined by routine experimentation depending on variables such as the size of the wound, the location of the wound, and the level and type of tissue damage. For systemic administration, a dose between 0.1 mg/kg and 100 mg/kg, alternatively between 0.5 mg/kg and 50 mg/kg, alternatively, between 1 mg/kg and 25 mg/kg, alternatively between 2 mg/kg and 10 mg/kg, alternatively between 5 mg/kg and 10 mg/kg, alternatively between 0.05-1.50 mg/kg is administered and may be given, for example, once daily, once weekly, twice weekly, three times weekly, once every other week, until complete healing of the injury is achieved.

### Pharmaceutical Formulations

For use in wound healing and promoting angiogenesis, the PRG4 administered is preferably combined with a pharmaceutically acceptable carrier. Pharmaceutically acceptable carriers include buffers, carriers, and excipients suitable for use in contact with the tissues of human beings and animals without excessive toxicity, irritation, allergic response, or other problem or complication, commensurate with a reasonable benefit/risk ratio. The carrier(s) should be “acceptable” in the sense of being compatible with the other ingredients of the formulations and not deleterious to the recipient. Pharmaceutically acceptable carriers include buffers, solvents, dispersion media, coatings, isotonic and absorption delaying agents, and the like, that are compatible with pharmaceutical administration.

For PRG4 delivered as a solution, suitable carriers include physiological saline, bacteriostatic water, Cremophor ELTM (BASF, Parsippany, N.J.) or phosphate buffered saline (PBS). Suitable carriers may also include a solvent or dispersion medium containing, for example, water, ethanol, polyol (for example, glycerol, propylene glycol, and liquid polyethylene glycol), and suitable mixtures thereof. The carrier should be stable under the conditions of manufacture and storage, and should be preserved against microorganisms. The use of carriers for pharmaceutically active substances is known in the art. For example, see Remington's Pharmaceutical Sciences, 18th ed. (Mack Publishing Company, 1990).

In some embodiments, pharmaceutical formulations of PRG4 are sterile. Sterilization of pharmaceutical preparations is achievable through known mechanisms.

The pH of the pharmaceutical preparations containing lubricin typically is between 3 and 11, more preferably between 5 and 9 or between 6 and 8, and most preferably between 7 and 8, such as 7 to 7.5.

For topical administration, lubricin may be incorporated into a paste, ointment, cream, lotion, gel, balm, or salve, or other suspension or emulsion. Such pharmaceuticals compositions may include water; oil; thickening agents such as cellulose, pectin, methylcellulose, or carbopol; buffering agents such as citrate buffer, phosphate buffer, or tartrate buffer; chelating agents such as EDTA or citric acid; emulsifying agents such as wax, cetostearyl alcohol, or polysorbate 20; humectants such as glycerin, glyceryl fatty acid esters, propylene glycol, or polyethylene glycol; permeation enhancers such as DMSO, urea, triethanolamide, alcohols, fatty acids, fatty acid esters, polyols, sodium lauryl sulfate, benzalkonium chloride, cetylpyridinium chloride, lecithins, Spans®, Tweens ®, poloxamers, miglyol®, or propylene glycol; and/or preservatives such as benzoic acid, alcohols, quaternary ammonium compounds or organic mercurial compounds such as thimerosal. Ointments, balms, or salves may include, for example, petroleum jelly or petrolatum, and may be made of an oleaginous base, an absorption base, a water in oil emulsion base, an oil in water emulsion base or a water soluble base. Creams and lotions may include water in oil or oil in water emulsions. Gels may include hydrogel or organogel bases including chemical or physical gels or single or two-phase systems. Suitable bases for these topical preparations may include petrolatum, white petrolatum, yellow or white ointment, mineral oil, lanolin, cholesterol, stearyl alcohol, polyethylene glycol, white wax, carbomer, carboxymethylcellulose, or hydroxy propyl methyl cellulose. Bases for topical preparations should be compatible with the skin, stable, smooth, pliable, non-irritating, and capable of absorbing water or other liquid preparations and of releasing the incorporated lubricin pharmaceutical active. Bases should be sterilizable. Preparation of paste, ointments, creams, lotions, gels, balms, or salves is known to the person of skill in the art.

For ocular administration, PRG4 may be incorporated into an ophthalmically acceptable formulation, for example, a solution or ointment or gel or collagen insert. Formulations may provide sustained release of PRG4. For example, a sustained release formulation of PRG4 for the eye may be achieved via a collagen insert.

According to some embodiments, a pharmaceutical preparation including lubricin and an antibiotic is provided. Examples of antibiotics that may be administered include amikacin, gentamicin, kanamycin, neomycin, netilmicin, tobramycin, paromomycin, streptomycin, spectinomycin, geldanamycin, herbimycin, rifaximim, loracarbef, ertapenem, doripenem, imipenem, cilastatin, meropenem, cefadroxil, cefazolin, cephradine, cephapirin, cephalothin, cephalexin, cefaclor, cefoxiting, cefotetan, cefamandole, cefmetazole, cefonicid, loracarbef, cefprozil, cefuroxime, cefixime, cefdinir, cefditoren, cefoperazone, cefotaxime, cefpodoxime, ceftibuten, ceftizoxime, moxalactam, ceftriaxone, cefepime, ceftaroline fosamil, ceftobiprole, teicoplanin, vancomycin, telavancin, dalbavancin, oritavancin, clindamycin, lincomycin, daptomycin, azithromycin, clarithromycin, erythromycin, roxithromycin, telithromycin, spiramycin, azetreonam, furazolidone, nitrofurantoin, linezolide, posizolid, torezolid, amoxicillin, ampicillin, azlocillin, dicloaxcillin, flucloxacillin, mexclocillin, methicillin, mafcillin, oxacillin, penicillin G, penicillin G, piperacillin, temocillin, ticarcillin, bacitracin, colistin, polymyxin B, ciprofloxacin, enoxacin, gatifloxacin, gemifloxacin, levofloxacin, lomefloxacin, moxifloxacin, nadifloxacin, nalidixic acid, norfloxacin, ofloxacin, trovafloxacin, grepafloxacin, sparfloxacin, temafloxacin, mafenide, sulfacetamide, sulfadiazine, sulfadimethoxine, sulfanilamide, sulfasalazine, sulfisoxazole, trimethoprim-sulfamethoxazole, sulfonamidochrysoidine, demeclocycline, doxycycline, metacycline, minocycline, oxytetracycline, tetracycline, clofazimine, dapsone, capremoycine, clycloserine, ethambutol, ethionamide, isoniazid, pyrazinamide, rifampicin, rifampin, rifaburin, rifapentine, streptomycin, arsphenamine, chloramphenicol, fosmomycin, fusidic acid, metronidazole, mupirocin, platensimycin, quinuprisitin/dalfopristin, thiamphenicol, tigecycline, tinidazole, or trimethoprim.

According to some embodiments, a pharmaceutical preparation including lubricin and an analgesic or anesthetic agent is provided. For example, the analgesic or anesthetic may be lidocaine, tetracaine, benzocaine, priolocaine, dibucaine, pramoxine, proparacaine, proxymetacaine, amethocaine, butamben, or oxybuprocaine.

### EXAMPLE 1

### Lubricin Expression in “Super Healer” MRL Mice

Applicants discovered that animals with spontaneous regenerative capacity (such as the MRL “super-healer” mouse) normally demonstrate an increase of lubricin expression after injury. This was determined using a mouse model as follows.

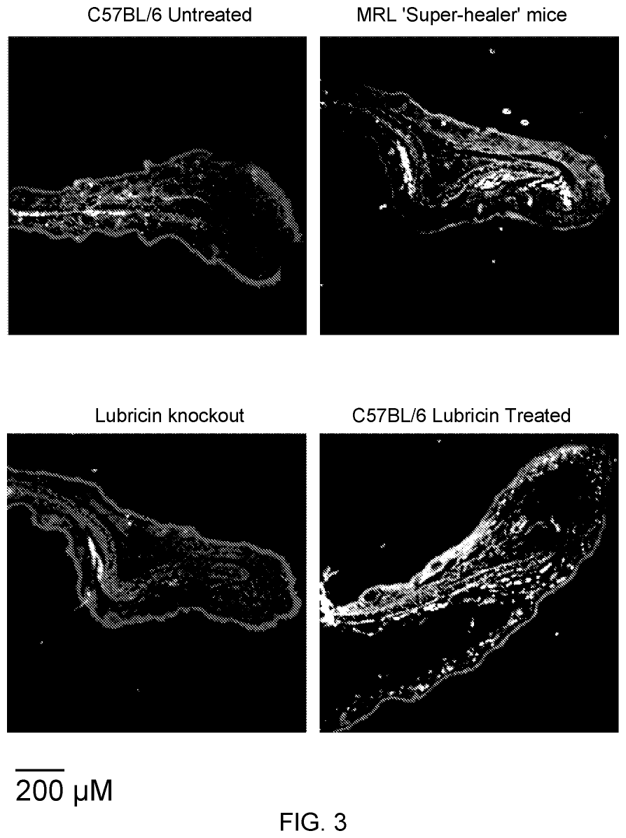

After receiving a 2 mm diameter punch injury to the ear, tissue samples from the ear of MRL mice, C57BL/6 control mice (untreated), lubricin treated C57BL/6 mice, and lubricin knockout mice were subjected to immunohistochemical staining to observe the presence of lubricin in the tissue 3 days after injury. As shown in FIG. 3, the green stain shows the presence of lubricin, whereas blue stain indicates regular cells without lubricin. As expected, no lubricin was present in the lubricin knock out mouse (bottom left panel) as evidenced by only blue stain, whereas in the MRL “super healer” mice, lubricin was abundant throughout the sample (top right panel) as demonstrated by dense green fluorescence throughout the image. In the C57BL/6 lubricin treated mice, lubricin is visible throughout the tissue (bottom right panel) as evidenced by an even mix of blue and green, although lubricin was not present to the same extent as in the MRL mice, whereas in untreated C57BL/6 mice, no lubricin was present (top left panel) as evidenced by only blue stain.

MRL mice also exhibited a regenerative response to injury with decreased scarring. This experiment demonstrates that increased lubricin staining correlates with increased wound healing and decreased fibrosis/scarring after injury.

These observations suggest that lubricin plays a role in regulating the repair (scar) versus regenerative response after injury.

### EXAMPLE 2

### Effects of Exogenous Lubricin in a Critical-Size Ear Wound in Mice

The ear is an ideal model to study regeneration, as it contains tissues from all three germ layers including epithelium, hair follicles, glandular tissue and cartilage. Accordingly, in order to observe the effects of exogenous lubricin on wound healing, a mouse model was used.

Each mouse was subject to a 2 mm diameter punch out of the ear on day 0. 24 mice were C57BL/6 mice while 12 were lubricin knock out mice (Prg4^{tm1Mawa}; Jackson Laboratories, Bar Harbor, Me.). Half of the C57BL/6 mice (n=12) were administered 25 μL lubricin topically to the punch site at a concentration of 100 μg/mL at the time of injury, and 1, 2, and 3 weeks post-injury. The other 12 C57BL/6 mice did not receive lubricin.

Effect on Observed Size of Wound

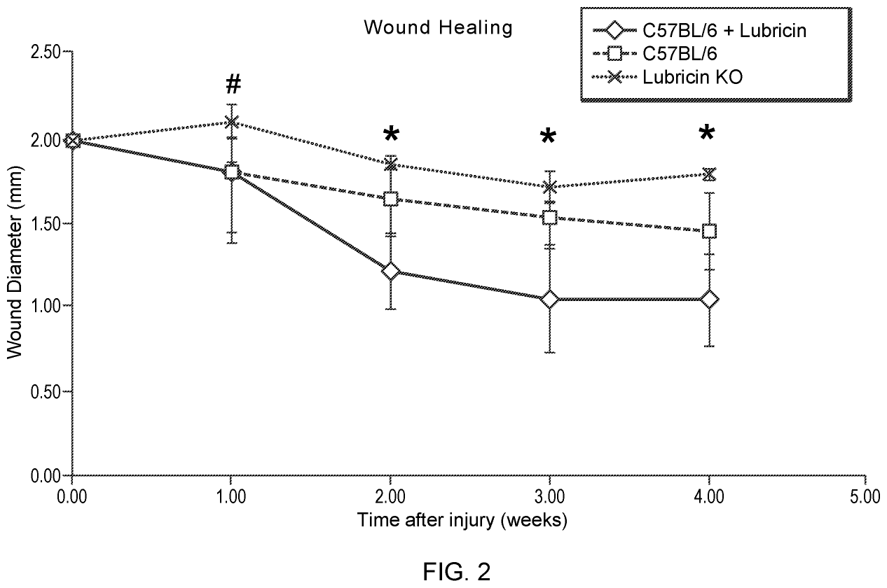

The mice were observed once weekly following the punch out and the size of the wound was measured by photographing the wound site with a known reference standard in each image. The results are shown in FIG. 2, which shows that by four weeks after injury, the average wound diameter in C57BL/6 mice who were administered lubricin was approximately 1.0 mm, half of the size of the punch out, whereas C57BL/6 mice who did not receive lubricin had a wound size averaging closer to 1.50 mm Knockout mice had the smallest decrease in wound size. This data shows that lubricin treatment significantly increases wound healing, while knocking out lubricin blocks wound healing.

Effect on Angiogenesis and Blood Flow at Injury Site

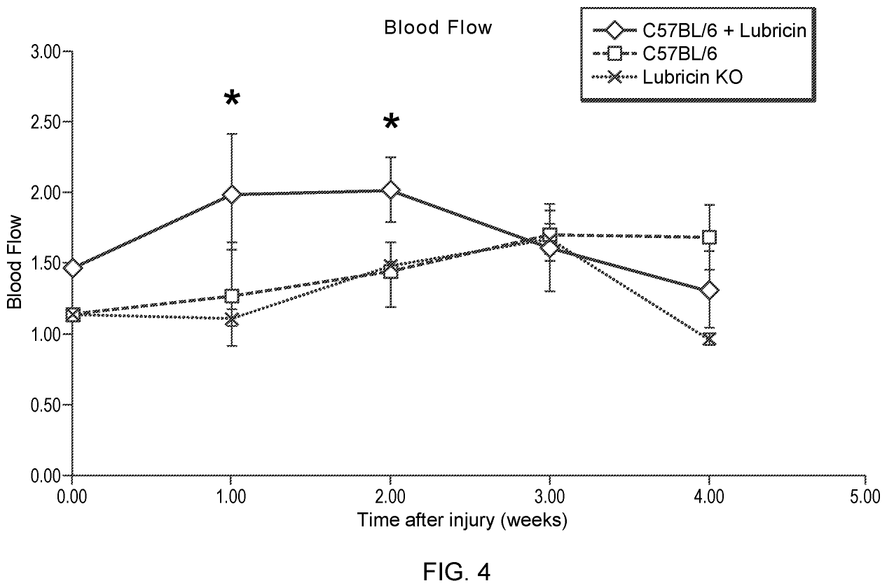

In order to determine whether lubricin had an effect on angiogenesis and blood flow at the injury site, blood flow at the ear injury site was measured in the C57B1/6 treatment and control mice as well as the lubricin KO mice. Measurements were obtained using laser speckle perfusion imaging (LSPI), a quantitative method using laser refraction speckle patterns to determine perfusion. Image exposure time was 15 msec. High-resolution images were processed using custom LSPI algorithms to create quantitative color perfusion maps measured in perfusion units (PU), an arbitrary unit defined and validated previously for this instrument (Forrester et al., (2002) Med Biol Eng Comput 40:687-697; Forrester et al., (2004) EEE Trans Biomed Eng 51:2074-2084. Blood flow was quantified once per week. As shown in FIG. 4, the level of blood flow at the injury site was significantly increased in C57BL/6 mice receiving lubricin over those mice that did not and over the lubricin KO mice, suggesting that administration of exogenous lubricin to a wound site significantly increases blood flow to the wounded tissue.

Given the observation of increased blood flow, mice were also tested for angiogenesis, i.e., the formation of new blood vessels. Angiogenesis was measured one week after injury using standard immunohistochemical staining and the number of positive vessels were quantified from those images.

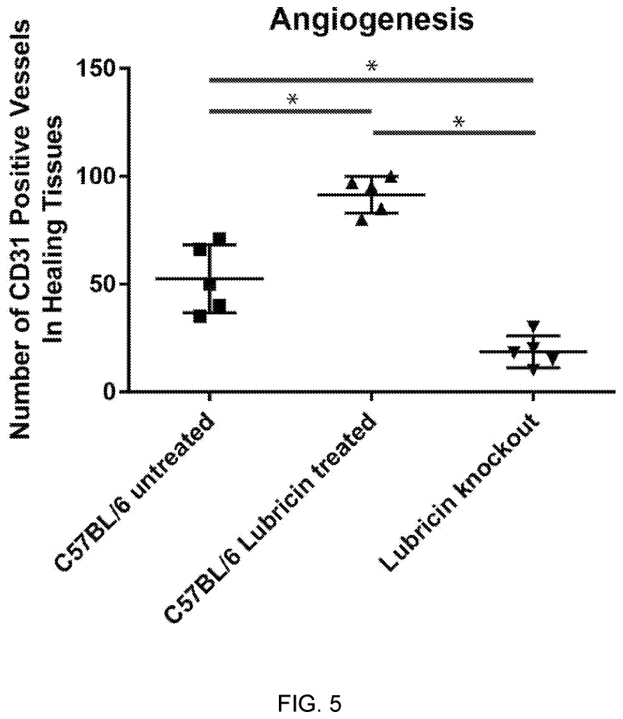

As shown in FIG. 5, increased number of CD31⁺ blood vessels were accounted for in healing tissues of the C57BL/6 mice receiving lubricin as compared to untreated C57BL/6 mice. The knockout mice had even fewer vessels, demonstrating that lubricin has a positive effect on angiogenesis in healing tissues.

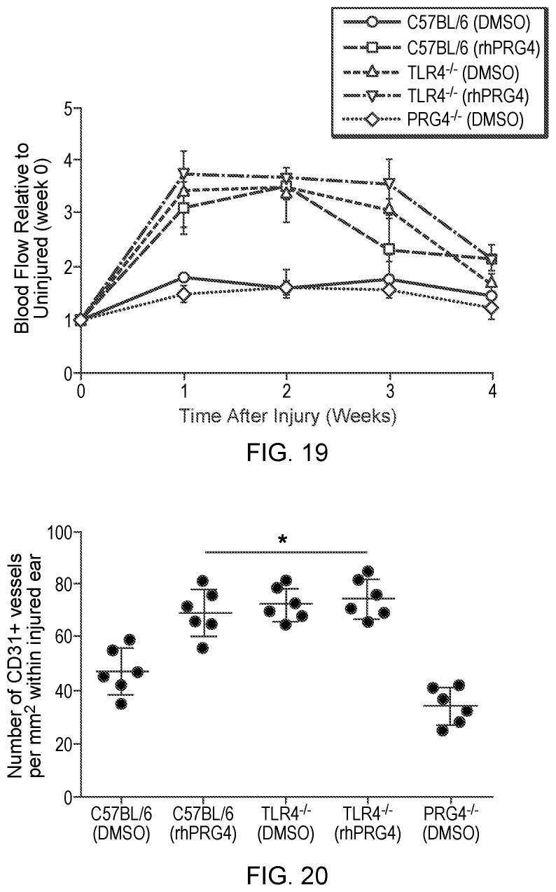

As shown in FIG. 19, rhPRG4 significantly increased blood flow to the tissue injury site as measured in mice injured by an ear punch wound measured 1, 2, 3, and 4 weeks after injury. As shown, TLR4−/− mice (treated with carrier or rhPRG4) and C57BL/6 mice treated with rhPRG4 had the highest levels of blood flow after injury.

Similarly, as demonstrated in FIG. 20, 1 week after a 4 mm² punch injury to the ear, increased numbers of blood vessels were observed in rhPRG4 treated mice and Tlr4−/− mice, while fewer vessels were observed in Prg4−/− animals, as measured by number of CD31+ vessels per mm² measured within the injured ear. The highest levels of angiogenesis at the injury site were observed in the PRG4 treated animals as well as the TLR4−/− animals

Effect on MSC Migration to the Wound Site

The role of stem cells in lubricin-mediated repair was also investigated. To follow the fate and characterize the response of the labelled cell populations, mice were induced with 4-hydroxytamoxifen (4-OHT, Sigma Aldrich) by intraperitoneal (i.p.) injections during 5 (five) consecutive days (1 mg per day) when mice were between 7-9 weeks of age. To fate map the Prx1-lineage MSCs (tdTomato^{+ve}) in the ear, samples were examined by histology/fluorescence imaging.

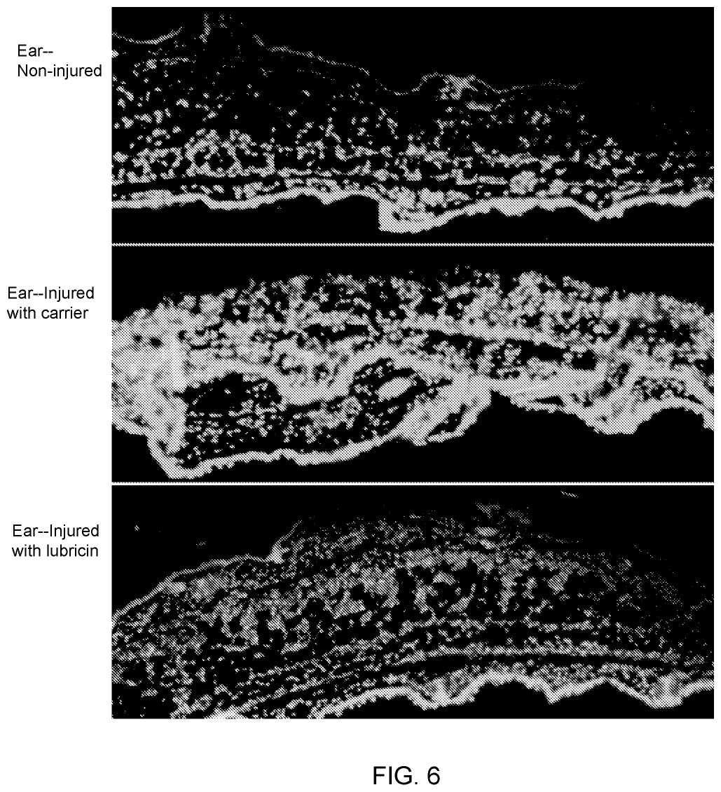

Treatment with lubricin was observed to result in mesenchymal stem cell (MSC) migration to areas of tissue injury. Within 1 week after injury, there was a substantial increase in MSCs observed at the injury site. However, only with exogenous lubricin treatment did these MSCs contribute to the regeneration of new ear (auricular cartilage). As shown in FIG. 6, essentially no MSCs were observed in non-injured ear tissue as evidenced by the absence of any red dots (top panel). However, MSCs were present in the tissue from injured ears where the wound was treated with carrier as a control (middle panel) and treated with lubricin (bottom panel). However, with lubricin treatment, MSCs completely repopulate the damaged cartilaginous structure of the ear, whereas without lubricin the MSCs were not presently in nearly the same amount. This data suggests that lubricin is involved in recruiting adult stem cells (MSCs) to the site of tissue injury to mediate tissue repair and that lubricin treatment is enhancing the potential of these cells above what is observed under normal wound healing conditions.

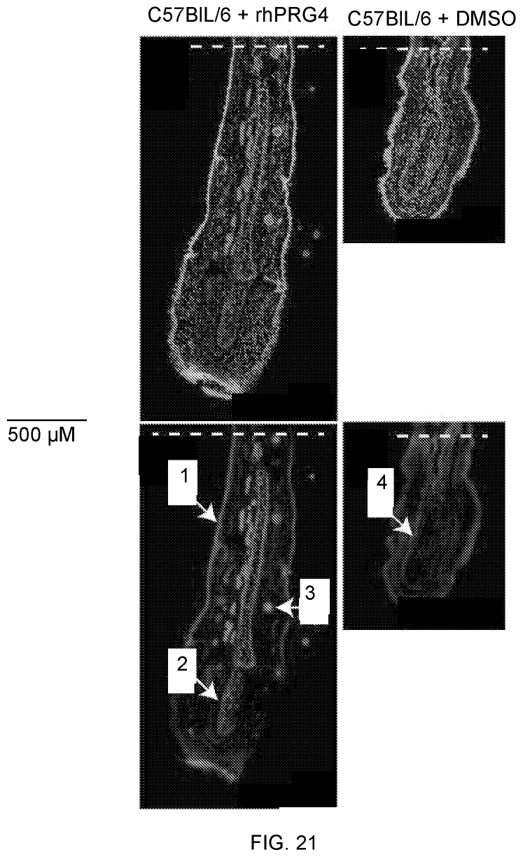

Further evidence that lubricin influences MSCs to promote wound healing is shown in the images in FIG. 21. Following full-thickness ear injury in MSC lineage reporter mice treated with rhPRG4, fate mapping of Tomato positive cells (MSCs) showed that they were the primary contributor to regeneration of cartilage and dermis and contributed to new hair follicles (FIG. 21, images on left hand side). This regenerative response was not observed in wild-type (C57BL/6) mice treated with vehicle (DMSO; images on right hand side), and interestingly lineage traced MSCs cells gave rise to disorganized fibrotic scar (FIG. 21; images on right hand side).

Effect on Recruitment of Immune Cells

Applicant examined macrophage populations and polarization after injury with lubricin treatment. To quantify cells present within the wound site, the mice were re-injured with a 2 mm through and through ear punch in the same area as the initial ear wound injury. This allowed collection of the tissue deposited from the initial time of injury. The ear tissue was dissociated using the gentleMACS™ Dissociator (Milteny, Bergisch Gladbach, Germany) according to the manufactures procedure. The resultant cell suspension was filtered and resuspended in 500 μl of 90% MeOH and left for 5-10 minutes at room temperature. The cells were then centrifuged, the liquid was removed and 500 μl of 0.1% Tween 20 was added to permeabilize the cells for 20 minutes at room temperature. The cells were centrifuged again, the liquid was removed, and 50 μl of Tween buffer and 0.5 μg of antibody was added to each tube and incubated in the dark for 30-45 minutes at room temperature. The cells were then washed three times with FACs buffer then resuspended in FACs buffer. The cells were then measured using FACs Caliber. The results were analyzed using FlowJo software.

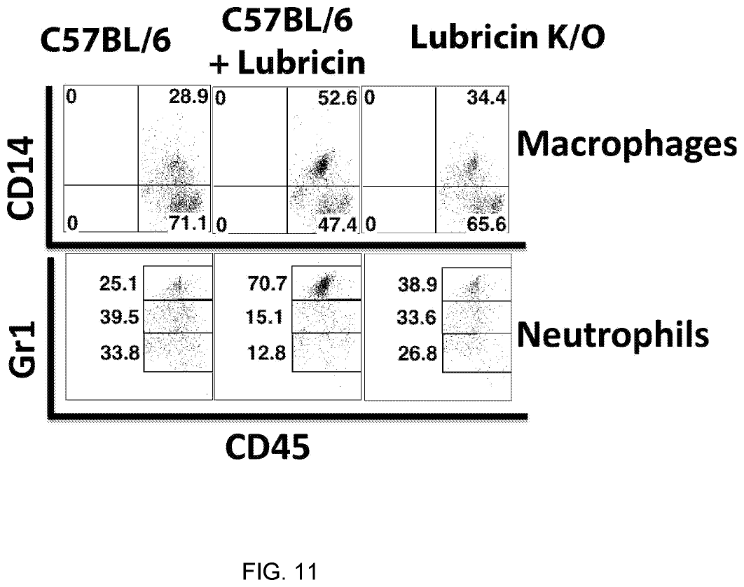

As shown by the flow cytometry analysis in FIG. 11, increased recruitment of both CD14⁺ macrophages and Gr1 neutrophils was observed in the injured ear after lubricin treatment compared to control and lubricin KO mice. In fact, approximately twice as many immune cells were found within the injury site in the lubricin treated C57BL/6 mice as compared to untreated C67BL6/mice and lubricin KO mice demonstrating that application of exogenous lubricin to the wound site results in an increased presence of macrophages and neutrophils at the site.

Possible Mechanism of Action

The data herein demonstrate that lubricin treatment can enhance endogenous repair of a critical sized defect within the ear by 1) inhibiting the fibrotic response, 2) increasing angiogenesis and blood flow to the injury, 3) regulating the inflammatory response (e.g. macrophage polarization) and 4) recruiting immune cells and adult stem cells (MSCs) to mediate tissue regeneration. The evidence provided herein shows that the absence of PRG4 critically impairs normal healing (as demonstrated by Prg4−/− KO Mice) and suggests that lubricin promotes a regenerative response by acting through at least three pathways to regulate wound healing, one acting at the level of angiogenesis and blood flow regulation at the injury site (NFκB/HIF1α/VEGF), one regulating the fibrotic response (PAI-1), and the last regulating the polarization of macrophages to either an M1 or M2 phenotype (TLR4). The evidence provided herein suggests that these pleiotropic effects are specifically attributable to lubricin since they do not occur in lubricin-deficient (KO) mice. These preliminary results in vitro and in vivo, suggest that lubricin suppresses the repair response (scarring) while enhancing the regenerative response (healing) after injury by modulating aspects of each of the conserved stages of wound healing: blood clotting, inflammation, tissue generation and tissue remodeling.

Results suggested that lubricin interacts with TLR4 on monocytes/macrophages to regulate the pro-inflammatory (M1) versus anti-inflammatory (M2) polarization of these immune cells. At the same time, lubricin also binds to PAI-1 to regulate the fibrotic response. Lubricin correspondingly regulates NFκB signaling which in turn upregulates HIF1αand VEGF expression to increase blood flow and angiogenesis within the wound area. We have also investigated the role of stem cells in lubricin-mediated repair and found that tissue-resident MSCs within the wound area express high levels of both lubricin and PAI-1 immediately after injury.

Lubricin-PAI-1 Interaction

Plasminogen activator inhibitor-1 (PAI-1) is a member of the serine protease inhibitor (serpin) gene family and an inhibitor of the serine proteases, uPA and tPA (Ghosh et al., J Cell Physiol. 2012, 227(2):493-507). Inhibition of uPA/tPA results in the inhibition of plasminogen-to-plasmin conversion as well as plasmin-dependent MMP activation. In normal wound healing, PAI-1 can be upregulated by TGF-β and then binds to and inactivates uPA and tPA (Botta et al., J Cell Sci. 2012, 125(Pt 18):4241-52). This inhibits activation of MMPs and tissue remodeling (clot stability). This leads to increased fibrin at the wound area and a corresponding increase in collagen 1 production by surrounding cells, producing in a fibrotic patch (scar formation) (Castro et al., J. Biol Chem., 2014, 289(42):29001-13).

Typically (except in the heart), animals deficient in PAI-1 demonstrate increased wound healing after injury with a decrease in fibrosis (Chan et al., Am J Pathol., 2001, 159(5):1681-8; Ghosh et al., PLoS One, 2013, 8(5):e63825) which is consistent with our hypothesis and preliminary data. However, a complete lack of PAI-1 can lead to substantial blood loss due to poor blood clot formation/stability and potentially, death (Iwaki et al., J Thromb Haemost., 2011, 9(6):1200-6.

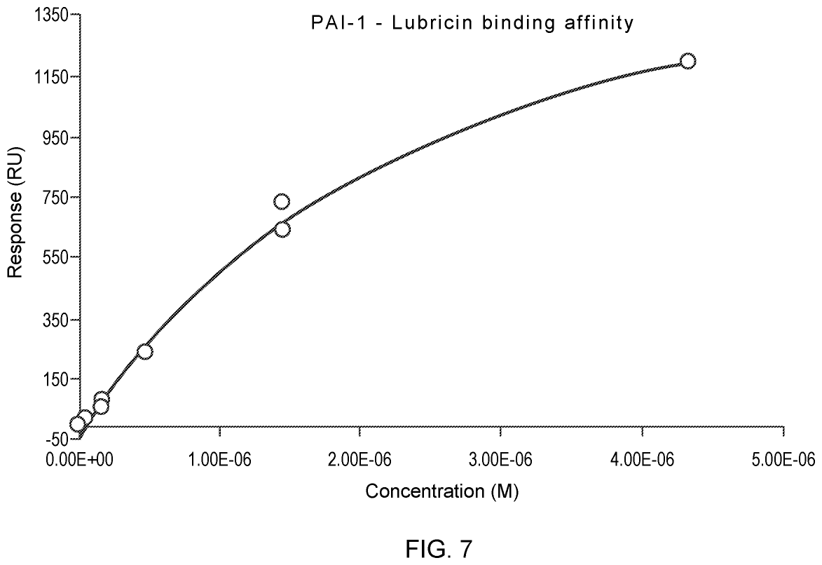

The data provided herein, as demonstrated in FIG. 7, show that lubricin binds directly to PAI-1. Binding of lubricin to PAI-1 was assessed using a Biacore X100 SPR instrument (GE Healthcare, Pittsburg Pa.). Human PAI-1 (R&D Systems, Minneapolis, Minn.) was immobilized onto the flow cell 2 of CMS sensor chip (GE Healthcare, Little Chalfont, United Kingdom) using standard amine-coupling chemistry, resulting in 300-500 response units (RU). The reference cell (flow cell 1) was prepared by activation and deactivation. The binding assay was performed in PBS running buffer supplemented with 0.01% (v/v) Tween 20. Lubricin solution was buffer exchanged to running buffer and at least 5 concentrations in the range of 0.576-420 μg/mL were injected at a flow rate of 30 μL/min with a contact time of 1 min at 25° C. The bound lubricin was removed from the chip surface by injecting 1 M NaCl after monitoring dissociation for 1.5 min. We determined that lubricin is able to bind directly to PAI-1 with a binding constant (K_{d}) of 2.712×10⁻⁶, which indicates strong affinity between the two molecules.

The interaction between PAI-1 and lubricin is likely due to the N-terminus somatomedin B (SMB) domain of lubricin which is nearly identical to the SMB domain within vitronectin (VTN), a known ligand of PAI-1 (Arroyo De Prada et al., Eur J Biochem. 2002, 269:184). VTN is known to play a pivotal role in regulating PAI-1 activity and stability (Jang et al., Surgery, 2000, 127(696); Zhou et al., Nat Struct Biol, 2003, 10:541). The data suggest that lubricin binding of PAI-1 acts as competitive inhibitor of PAI-1 signaling, inhibiting its activity to the point where increased wound healing and decreased fibrosis are observed, but not to the extent where clot formation is inhibited and chronic blood loss is observed.

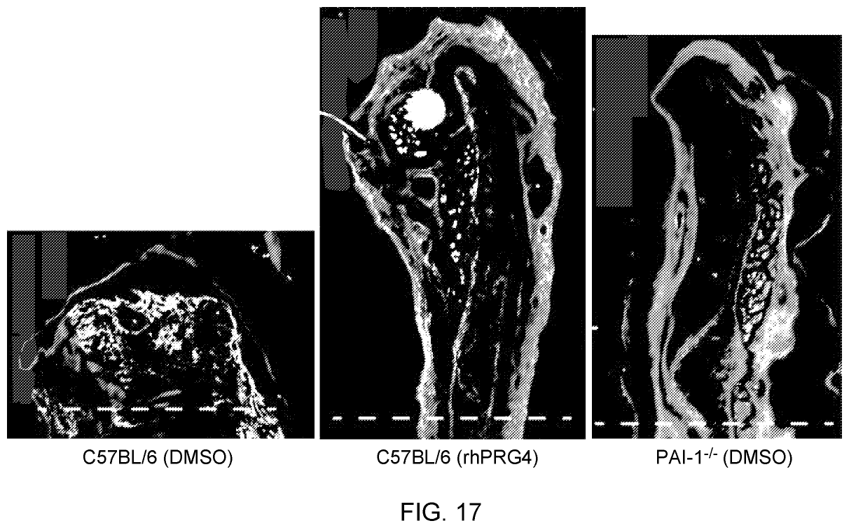

Further, as shown in FIG. 17 ear injuries in Pai-1^{-/-} (DMSO treated) and C57BL/6 mice (rhPRG4 treated) mice demonstrate significantly less a-smooth muscle actin (α-SMA, fibrotic marker) expression compared to C57BL/6 mice (DMSO treated. The images in FIG. 17 were taken at four weeks post-injury in an ear punch wound model in mice. The C57BL/6 mice show robust αSNA staining (white) while reduced αSMA staining is observed in C57BL/6 rhPRG4 treated and PAI-1−/− mice.

### Lubricin-NFκB/HIF1αNEGF

The data provided herein suggest that lubricin regulates angiogenesis through activation of VEGF via a HIF1a dependent pathway potentially upstream and/or downstream of NFκB (Fitzpatrick et al., J Immunol. 2011, 186(2):1091-6). Vascular endothelial growth factor (VEGF) is a highly specific mitogen for vascular endothelial cells: animals that lack even one of the two VEGF alleles die before birth because of defects in the development of the cardiovascular system (Haiko et al., Mol Cell Biol. 2008 28(15):4843-50). Hypoxia-induced VEGF production stimulates the angiogenesis that accompanies organ formation during development. Thus HIF1α (Hypoxia-inducible factor) is well known in the literature as a potent activator of VEGF, typically in areas of low oxygen (van Tuyl et al., Am J Physiol Lung Cell Mol Physiol. 2005 288(1):L167-78; Arany et al., Proc Natl Acad Sci U S A. 1996 93(23):12969-73).

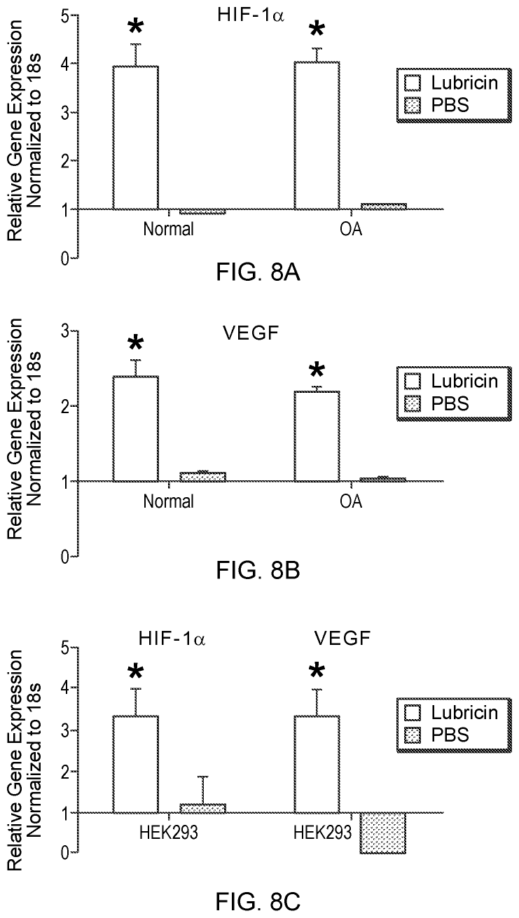

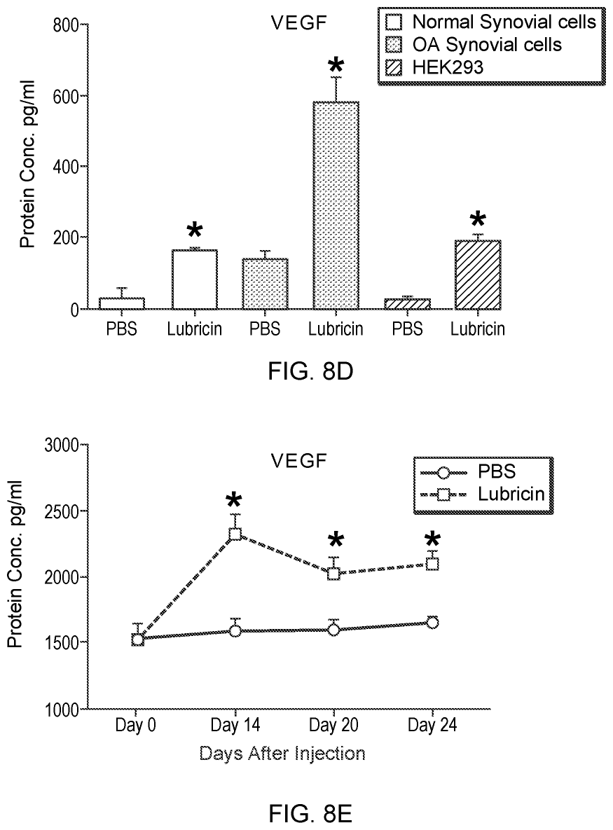

As shown by the data in FIGS. 8A-E, Lubricin up-regulates HIF1a and VEGF in multiple cell types and in vivo. In order to demonstrate this effect in vitro, synovial fibroblasts from normal and osteoarthritic (OA) human joints were exposed to lubricin (100 μg/mL; 2 mL) (FIGS. 8A-B), and after 24 hours, measurements were taken . In normal and OA cells, administration of exogenous lubricin significantly up-regulated expression of HIF1a (FIG. 8A) and VEGF (FIG. 8B) mRNA. This observation was validated in HEK293 cells, where it was observed that HIF1a and VEGF mRNA were both up-regulated 24 hours after lubricin treatment (100 μg/mL; 2 mL) (FIG. 8C).

As shown in FIG. 8D, ELISA was used to validate the levels of VEGF protein in normal synovial cells, osteoarthritic synovial cells, and HEK293 cells exposed to PBS carrier (control) or lubricin (100 μg/mL; 2 mL). In each type of cells, the presence of lubricin significantly upregulated the levels of VEGF over controls.

The data in FIG. 8E further demonstrate the in vivo effect of exogenously administered lubricin on increasing VEGF protein levels. In this experiment, 5 Sprague Dawley rats were injected with lubricin (200 μg/kg) via tail vein injection; and VEGF levels in serum were assayed using ELISA at days 0, 14, 20, and 24. As shown in FIG. 8E, VEGF was significantly up-regulated systemically 14-24 days after lubricin injection as compared to VEGF levels in rats that did not received lubricin, which demonstrated a steady level of VEGF expression.

Further, this lubricin-VEGF pathway is HIF1α dependent, since cells treated with HIF1α inhibitors demonstrate no increase in VEGF expression after lubricin treatment.

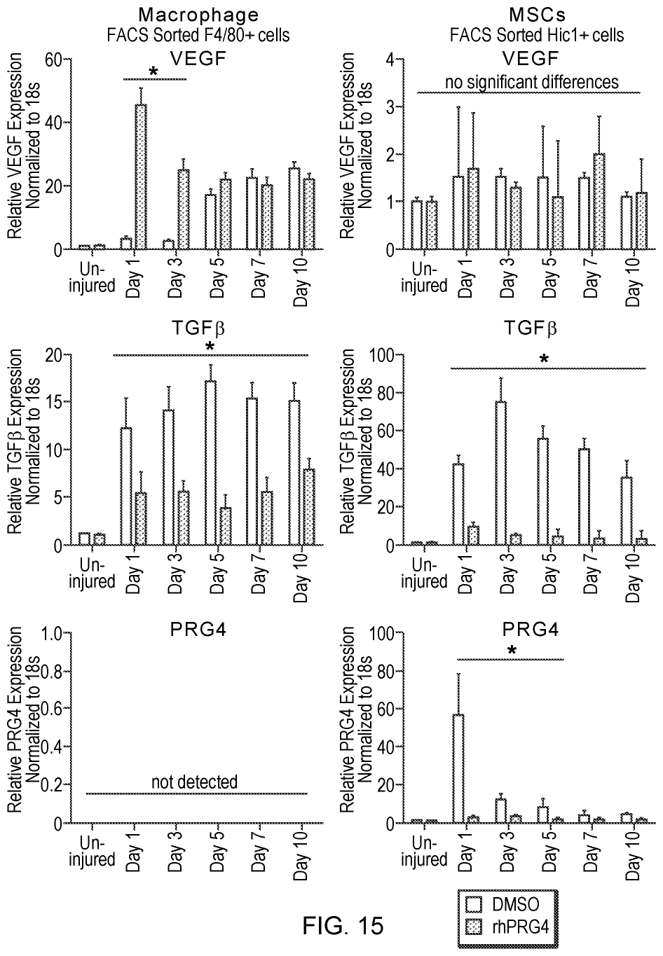

In addition, we isolated macrophages (F4/80⁺) and mesenchymal stem cells (MSC) from ear wounds treated with or without rhPRG4. We observed that rhPRG4 significantly increased vascular endothelial growth factor (Vegf) expression in macrophages but not MSCs, and also significantly decreased Tgfβ expression in macrophages and MSCs, as shown in FIG. 15. These results also demonstrate Prg4 expression is upregulated in MSCs post-injury, and that exogenous rhPRG4 inhibits the expression of endogenous Prg4 (negative feedback). Furthermore, while it is known that TGFI3 upregulates Prg4 (Schmidt et al., Osteoarthr. Cartil. 2008, 16, 90; Jones et al., Eur. Cell. Mater, 2007, 13;40), we now show there is a negative feedback loop between rhPRG4 and Tgfβ expression.

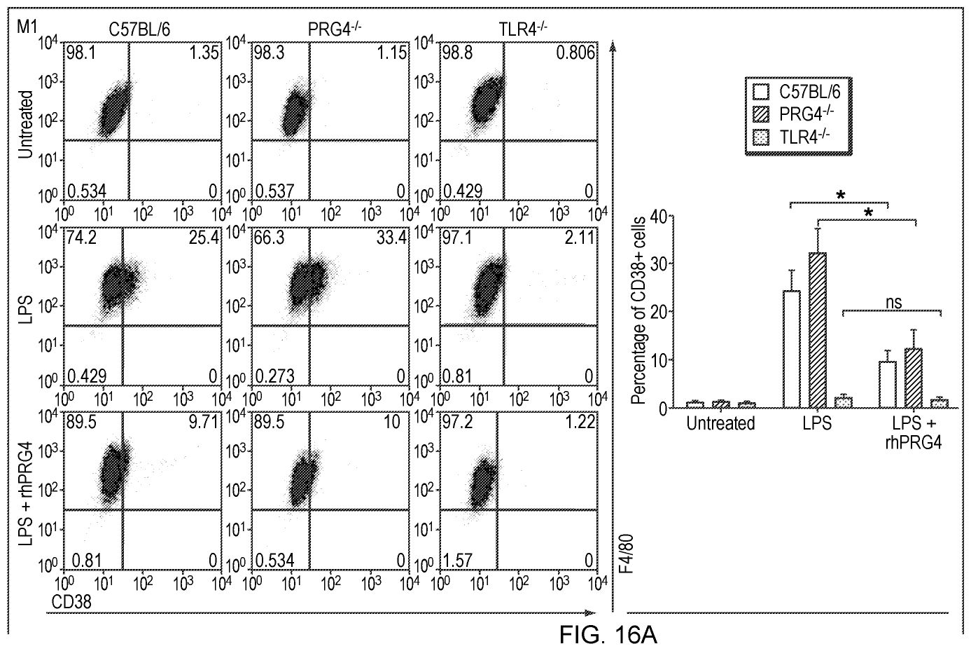

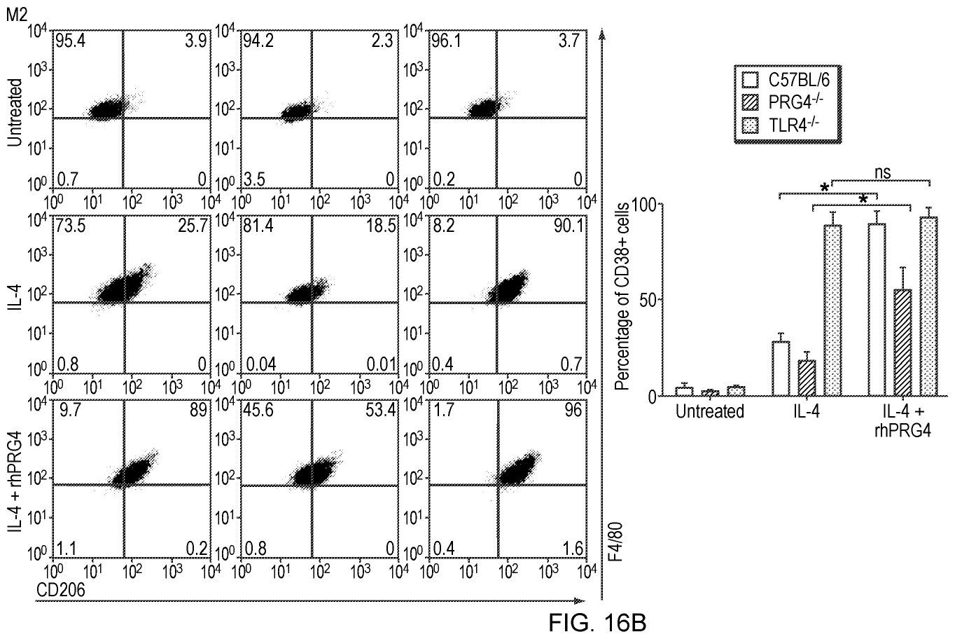

It has been previously shown that Tlr4^{-/-} macrophages have an M2 polarization bias (On et al., Diabetes 2012, 61:2718), and we found that C57BL/6 macrophages treated with rhPRG4 also demonstrate an M2 bias (FIG. 16A-B). Since an increase in VEGF is associated with anti-inflammatory (M2) polarization of macrophages (Lai et al., J. Cell. Mol. Med., 2018, 23:14027; Wheeler et al. PLoS One 2018, 13: e0191040), we isolated bone marrow monocytes from C57BL/6 and Tlr4^{-/-} mice and polarized them towards an M2 phenotype.

Monocytes were isolated from mice and differentiated into macrophages (M0); they were then polarized into pro-inflammatory macrophages with LPS (M1) or anti-inflammatory macrophages with IL-4 (M2). The results provided in FIGS. 16A-B show that C57BL/6 monocyte derived macrophages display diminished M1 polarization and enhanced M2 polarization in the presence of rhPRG4. Macrophages from Prg4^{-/-} mice demonstrate enhanced M1 polarization and diminished M2 polarization, which can be rescued by rhPRG4 treatment and show that PRG4 or the lack of PRG4 regulates the polarization of macrophages. These affects appear to be TLR4-dependent as Tlr4^{-/-} macrophages demonstrate diminished M1 polarization and enhanced M2 polarization, which is not altered by rhPRG4 treatment. Furthermore, treating Tlr4^{-/-} macrophages with rhPRG4 had no additive effect, suggesting that the ability of rhPRG4 to regulate polarization is TLR4-dependent.

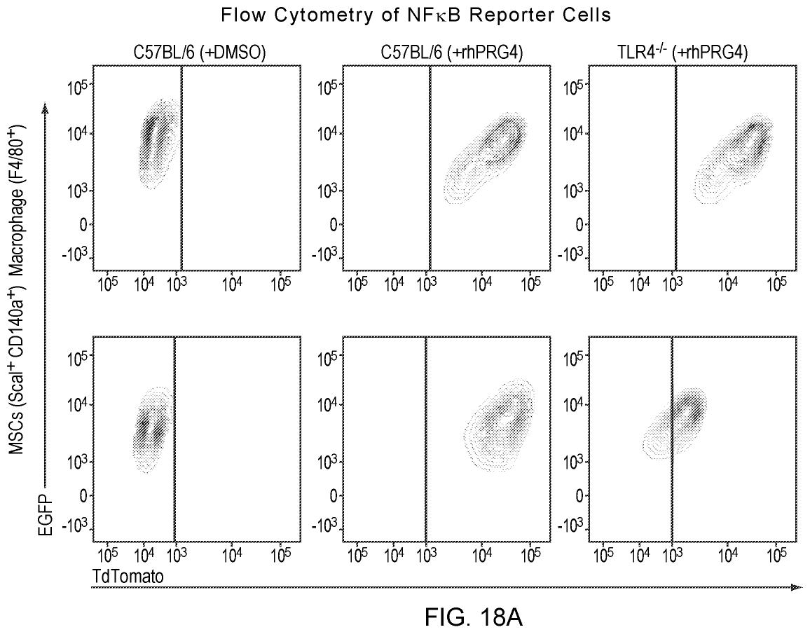

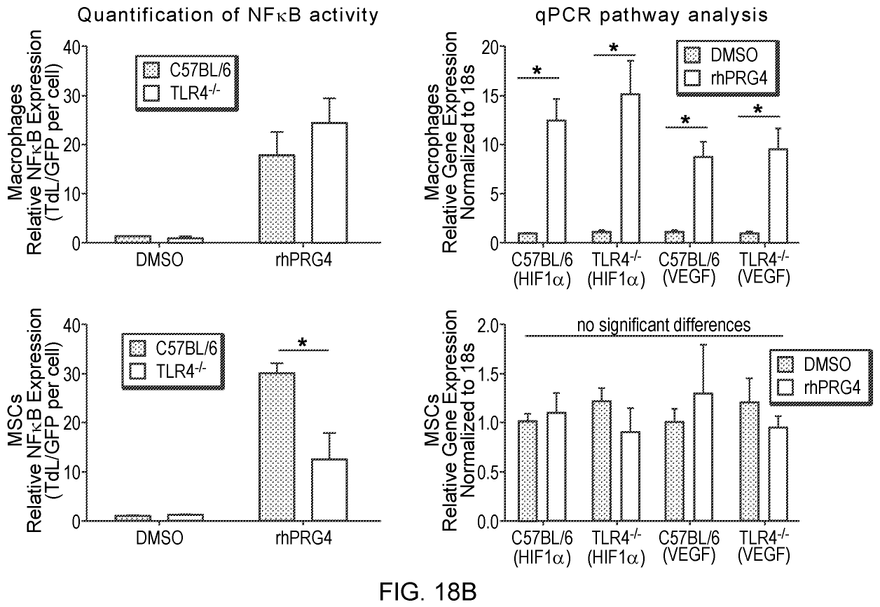

In addition, we have determined that rhPRG4 treatment also upregulates NFκB activity, as shown in FIGS. 18A-B. Macrophages and MSCs were isolated from non-injured mice and NFκB activation was monitored through a tdTomato reporting vector. The data, presented in FIGS. 18A-B, show that rhPRG4 treatment upregulates NFκB expression in macrophages and MSCs from non-injured mice. This appears to occur in independent of TLR4 since the same response was observed between TLR4−/− mice and the C57BL/6 mice. We have previously shown that rhPRG4 regulates NFκB signaling both in vitro and in vivo (Iqbal et al., 2016, Sci Rep., 6: 18910) and through the use of an NFκB reporter, we have demonstrated, as shown in FIGS. 18A-B, a concurrent activation of NFκB and Hif1α and Vegf post-rhPRG4 treatment. However, only macrophages, not MSCs, showed increased Hif1α and VegF expression suggesting that the pro-angiogenic response upon exposure to PRG4 is driven by macrophages and not MSCs.

FIGS. 18A-B also suggest that that rhPRG4 regulates angiogenesis through activation of VEGF via HIF1α. This potentially occurs downstream of NFκB (Fitzpatrick, et al., J. Immunol., 2011, 186:1091). Hypoxia-induced VEGF production stimulates the angiogenesis that accompanies organ formation during development. Thus HIF1α (Hypoxia-inducible factor) is well known in the literature as a potent activator of VEGF, typically in areas of low oxygen (Van Tuyl et al., Am. J. Physiol. Lung Cell. Mol. Physiol. 2005, 288:L167). In vitro data in FIGS. 18A-B and ex vivo data in FIG. 15 show that treatment of macrophages with rhPRG4 increases Vegf at the mRNA level.

Lubricin-TLR4 Interaction

TLRs are an important class of pattern-recognition receptors expressed predominantly by macrophages involved in the innate immune system. Once TLRs are activated, the innate immune cell response is triggered (Parker et al., Clin Exp Immunol. 2007; 147:199-207, Zhang et al., Cell Mol Life Sci. 2006; 63:2901-7). TLRs recognize highly conserved motifs known as pathogen-associated microbial patterns (PAMPs), which are expressed by pathogens, or danger-associated molecular patterns (DAMPs/Alarmins) that are released from necrotic or dying cells (Rickard et al., Cell. 2014; 157:1175-88). TLR4 is required for normal wound healing (Suga et al., J Dermatol Sci. 2014 73(2):117-24; Dasu et al., J Diabetes Complications. 2013 27(5):417-21) and is required for Ml versus M2 polarization, as mice lacking TLR4 showing a bias towards an M2 (or anti-inflammatory) phenotype (On et al., Diabetes. 2012 61(11):2718-27)

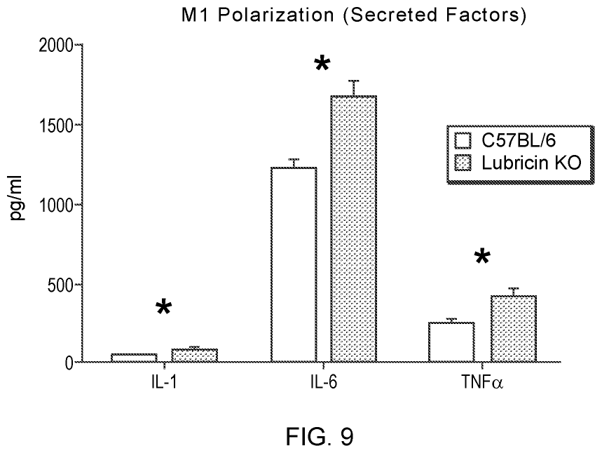

We examined macrophage polarization in lubricin-deficient mice. As shown in FIG. 9, the macrophages of lubricin-deficient (KO) mice display an M1 (pro-inflammatory) polarization bias and produce significantly more IL-1, IL-6 and TNFα than wild-type control mice. In other words, polarization of macrophages in lubricin knock out mice leads to an increase of pro-inflammatory factors compared to wild-type controls. Given that TLR4 knock out animals' macrophages demonstrate an M2 bias (Gupta et al., Biochem Biophys Res Commun. 2016 477(3):503-8), this suggests that the presence of lubricin at least partially inactivates TLR4, whereas absent lubricin, TLR4 activation leads to pro-inflammatory polarization of macrophages.

Further, in data displayed in FIGS. 16A-B, we determined that treatment of macrophages with PRG4 inhibits M1 and enhances M2 polarization and is TLR-4 dependent. FIGS. 16A-B show that macrophage polarization in Prg4^{-/-} mice display an M1 (pro-inflammatory) polarization bias, while C57BL/6 macrophages treated with rhPRG4 demonstrate an M2 bias.

PAI-1-VEGF-TLR4 Signaling

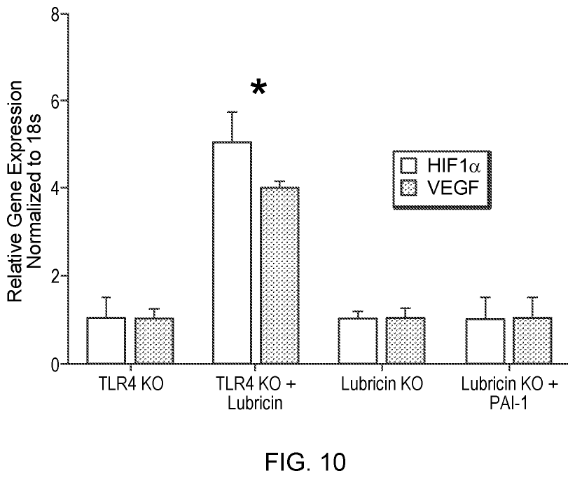

The PAI-1, VEGF, and TLR4 signaling pathways interact with each other at multiple levels (Siegel-Axel et al., Diabetologia. 2014 57(5):1057-66; Wu et al., Am J Pathol. 2014 184(6):1900-10; Biernaskie et al., Cell Stem Cell. 2009 5(6):610-23), our data suggests that each one of these pathways is independent in vitro. As shown in FIG. 10, an increase in VEGF transcription after lubricin treatment was TLR4 independent since TLR4 knockout cells still express VEGF after lubricin treatment. Additionally, as demonstrated in FIG. 10, when lubricin knockout cells were treated with recombinant PAI-1, no increase in VEGF was observed, suggesting that VEGF upregulation after lubricin treatment is independent of PAI-1.

PRG4 Mediated Regenerative Response Acts through TLR4 and PAI-1

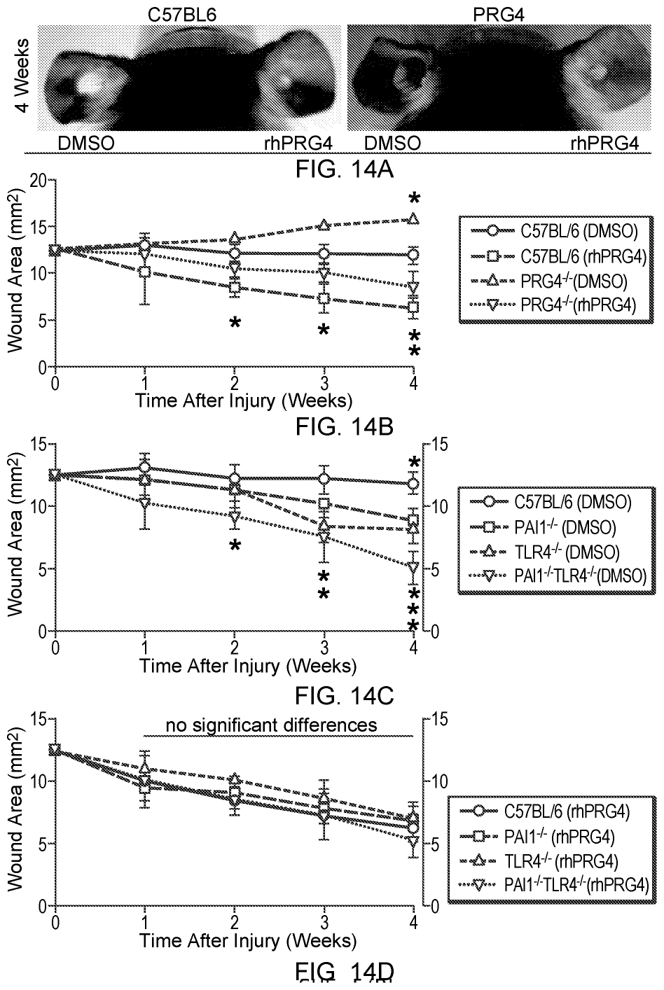

Data described in FIGS. 14A-D clearly demonstrates that rhPRG4 enhances wound healing when applied topically, and the absence of Prg4 critically impairs normal healing (FIG. 14A). Results from the mouse ear wound model described herein, suggest that this PRG4 mediated regenerative response is acting through TLR4 and Plasminogen Activator Inhibitor-1 (PAI-1). Specifically, Tlr4^{-/-} and Pai-1^{-/-} mice present with improved ear wound healing (relative to C57BL/6), and exogenous delivery of rhPRG4 further enhances this effect in both these strains (FIG. 14C-D). Furthermore, Tlr4 Pai-1^{-/-} double knockouts demonstrate the same regenerative response as rhPRG4 treated C57BL/6 mice. Yet, the addition of rhPRG4 to Tlr4^{-/-}Pai-1^{-/-} mice has no additive effect on ear wound healing. (FIG. 14D). This suggests that TLR4 and PAI-1 signaling are negative regulators of ear wound healing and rhPRG4 treatment in C57BL/6 mice inhibits these signaling pathways to increase regenerative potential.

### EXAMPLE 4

### Treatment of Wounds in a Human Patient Diabetic Ulcer

A human patient presents with a diabetic ulcers on several toes. Each ulcer is examined and cleaned. A solution containing recombinant human lubricin in an amount of 100 μg/mL is applied to each ulcer as a series of drops in sterile phosphate buffered saline. 80 μg of lubricin is applied per square centimeter of ulcerated tissue. Once the solution dries, an appropriate bandage is placed on each ulcer. The treatment is repeated weekly for four weeks. After treatment is completed, the ulcers have healed and the tissue has regenerated. There is no noticeable scar tissue at the formerly injured site.

### Epidermal Abrasions

A human patient presents with abrasions, commonly known as “road rash” over the arms, legs, and stomach after a biking accident. The wounds are cleaned and a sterile gel comprising recombinant human lubricin is applied topically to the abraded skin and covered with appropriate bandages. At least 80 μg of lubricin is applied per square centimeter of wounded tissue. The treatment is repeated weekly for four weeks, and upon reapplication, the lubricin ointment is applied only to the remaining areas of tissue that have not yet healed. Accordingly, each week, less tissue requires application of the lubricin ointment. When the road rash finally heals, no visible scarring is present.

### Cut or Laceration

A human patient presents with a 3 cm laceration on the index finger of the right hand. The laceration is cleaned and an ointment containing recombinant human lubricin is applied to the laceration, prior to stitching the laceration with several sutures. A bandage is applied. Each day ointment containing lubricin is reapplied to the affected area in the amount of about 80 μg per cm length of the injury. After one week the stitches are removed. Lubricin ointment is applied daily to the wound for another week until the stitch marks have disappeared. No scar formation at the site of the laceration is observed.

### Ophthalmic Injury

After receiving a photorefractive keratectomy, a human patient develops a corneal haze in one eye. The patient is prescribed an eye drop containing PRG4. The patient applies 2 0.05 mL drops of 100 μg/mL PRG4 to the cul de sac of the eye 4 times daily, applying the PRG4 to the corneal surface by rapid blinking After one week, the corneal haze has cleared and the patient's vision is no longer cloudy.

1. A method of promoting tissue regeneration or wound healing while reducing scar formation comprising administering to a wound or the site of tissue injury in a patient in need thereof a pharmaceutical preparation comprising lubricin.

2. The method of claim 1, wherein the lubricin is recombinant human lubricin.

3. The method of any one of claims 1-2, wherein the lubricin has the amino acid sequence of SEQ ID NO:1 or residues 25-1404 of SEQ ID NO:1.

4. The method of any one of claims 1-3, wherein the lubricin has at least 95% amino acid sequence identity with the sequence of SEQ ID NO:1 or residues 25-1404 of SEQ ID NO:1.

5. The method of any one of claims 1-4, wherein the wound or tissue injury is in the eye.

6. The method of any one of claims 1-5, wherein the wound or tissue injury is in the cornea.

7. The method of any one of claims 1-4, wherein the wound is present in the epidermis, dermis, and/or hypodermis layer(s) of the skin.

8. The method of any one of claims 1-7, wherein the wound is a cut, puncture, laceration, tear, scrape, or abrasion to the skin, or a surgical incision.

9. The method of any one of claim 1-4 or 7, wherein the wound is a bruise.

10. The method of any one of claim 1-4 or 7, wherein the wound is a burn.

11. The method of any one of claim 1-4 or 7, wherein the wound is a dermal ulcer.

12. The method of claim 11, wherein the ulcer is decubitus (pressure ulcer), a diabetic ulcer, or an ulcer caused by bacterial infection.

13. The method of any one of claims 1-12, wherein the wound is at a site that is non-articular, non-osseous, and non-osteal and the wound is not in a bone, joint, articular cartilage, tendon or ligament.

14. The method of any one of claims 1-13, wherein the lubricin is provided at a concentration of 1 μg/mL to 1 mg/mL.

15. The method of claim 14, wherein the lubricin is provided at a concentration of 100 μg/mL.

16. The method of any one of claims 1-15, wherein the lubricin is provide in an amount of 10-150 μg per cm² of wound area.

17. The method of claim 16, wherein the lubricin is providing in the amount of 75-100 μg per cm² of wound area.

18. The method of any one of claims 1-17, wherein the pharmaceutical preparation is administered as a solution, suspension, emulsion, lotion, cream, gel, paste, or ointment.

19. The method of claim 18, wherein the pharmaceutical preparation is applied topically to the wound.

20. The method of any on claims 1-19, wherein the pharmaceutical preparation is administered as an impregnate of a wound dressing that is applied to the wound.

21. The method of any one of claims 1-20, wherein the pharmaceutical preparation further comprises an antibiotic or an anti-inflammatory agent or an analgesic agent or is administered with an antibiotic, anti-inflammatory, or analgesic agent.

22. The method of claim 21, wherein the analgesic agent is lidocaine or benzocaine.

23. The method of claim 22, wherein the antibiotic is neomycin, polymyxin b, bacitracin, erythromycin, retapamulin, sulfacetamide sodium, mupirocin, pramoxine, silver sulfadiazine, mafenide, or ozenoaxacin.

24. The method of any one of claims 1-23, comprising administering said pharmaceutical preparation as liquid drops or by injection at the site of the wound.

25. The method of any one of claims 1-24, wherein the pharmaceutical preparation does not include hyaluronic acid.

26. A pharmaceutical composition for use in promoting tissue regeneration or wound healing while reducing scar formation comprising lubricin.

27. A method of inducing angiogenesis at a site in a body of a patient in need thereof, comprising administering a pharmaceutical preparation comprising lubricin to the site in an amount sufficient to induce new blood vessel formation.

28. The method of claim 27, wherein the lubricin is recombinant human lubricin.

29. The method of any one of claims 27-28, wherein the lubricin has the amino acid sequence of SEQ ID NO:1 or residues 25-1404 of SEQ ID NO:1.

30. The method of any one of claims 27-29, wherein the lubricin has at least 95% amino acid sequence identity with the sequence of SEQ ID NO:1 or residues 25-1404 of SEQ ID NO:1

31. The method of any one of claims 27-30, wherein the lubricin is provided at a concentration of 1 μg/mL to 1 mg/mL.

32. The method of claim 31, wherein the lubricin is provided at a concentration of 100 μg/mL.

33. The method of any one of claims 27-32 wherein the lubricin is provided in an amount of 10-150 μg per cm² area of the site.

34. The method of any one of claims 27-32, wherein the lubricin is provided in the amount of 75-100 μg per cm² area of the site.

35. The method of any one of claims 27-34, wherein the lubricin is administered topically to the site.

36. The method of any one of claims 27-34, wherein the lubricin is administered to the site by injection.

37. The method of any one of claims 27-36, wherein the pharmaceutical preparation does not include hyaluronic acid.

38. The method of any one of claims 27-37 wherein the site in the body is the skin or the eye excluding the cornea.

39. The method of any one of claims 27-38, wherein the site in the body is non-articular, non-osseous, and non-osteal and the site is not in a bone, joint, or articular cartilage

40. A pharmaceutical composition for use in promoting angiogenesis comprising lubricin.

41. The pharmaceutical composition of claim 26 or 40 further comprising a pharmaceutically acceptable carrier.

42. A method of treating or preventing aberrant wound healing in an eye comprising administering lubricin to an eye suffering from or at risk of aberrant wound healing.

43. The method of claim 42, wherein the aberrant wound healing is scarring of the cornea.

44. The method of claim 42, wherein the aberrant wound healing is corneal haze.

45. The method of any one of claims 42-44, wherein the lubricin is provided at a concentration of 1 μg/mL to 1 mg/mL.

46. The method of any one of claims 42-45, wherein the lubricin is provided in an amount of 10-150 μg per cm² based on the size of the wound.

47. The method of any one of claims 42-46, wherein the lubricin has at least 95% amino acid sequence identity with the sequence of SEQ ID NO:1 or residues 25-1404 of SEQ ID NO:1.

48. The method of any one of claims 42-47, wherein the lubricin is administered ophthalmically as drops or an ointment.

49. The method of any one of claims 42-48, wherein the lubricin is administered to the eye upon completion of a keratinoplasty, photorefractive keratectomy, laser subepithelial keratomileusis, or laser in situ keratomileusis.
